(19) United States
(12) Patent Application Publication
(10) Pub. No.: US 2018/0122497 A1
(43) Pub. Date: May 3, 2018

(54) Systems, Methods and Data Structures for Efficient Processing and Presentation of Patient Medical Data

(71) Applicant: Celgene Corporation, Summit, NJ (US)

(72) Inventors: Dennis Pietronigro, Clinton, NJ (US); Jianping Huang, Cranford, NJ (US)

(21) Appl. No.: 15/575,657

(22) PCT Filed: May 20, 2016
(86) PCT No.: PCT/US2016/033433

§ 371 Date: Nov. 20, 2017

Related U.S. Application Data
(60) Provisional application No. 62/164,955, filed on May 21, 2015.

(51) Int. Cl. G16H 10/20; G16H 10/60; G06F 17/21

(52) U.S. Cl. CPC G16H 10/20; G06F 17/246; G06F 17/211; G16H 10/60

(57) ABSTRACT

Systems, methods and data structures for transforming patient data from source computer data structures into another format of a different computer data structure for efficient processing and visualization of patient data for medical treatment, e.g., a clinical trial, are described. Source data of various source data structures for the patients is selected, where the source data is arranged among data fields, each of which has a data type and a content type. Selected source data is assigned to multiple data fields of a first data structure to populate the patient data of the first data structure, wherein the assigning includes transforming source data of different content types for two or more fields of the other data structures into a singular, particular transformative data field of the first data structure. The multiple data fields of the first data structure for a given patient include multiple sets of data fields for multiple treatments at different times. The patient data of the first data structure is processed for visualization at a graphical user interface such that representations of the multiple data fields of the first data structure for the given patient are arranged for display via a single page of a graphical user interface (GUI), and such that certain data fields of the multiple fields of the first data structure are structured so as to not visibly convey numerical or textual data via the graphical user interface, wherein at least some of the certain data fields convey substantive information by virtue of not displaying visible numerical or textual data. In some examples, certain patient data within normal ranges is not assigned to the first data structure or displayed in the single page of the GUI. This can result in a compact representation and visualization of the patient data for any given patient that avoids presenting the voluminous amount of ordinary information and normal-range data to permit a physician to quickly comprehend the most important information.

This application claims the benefit of U.S. Provisional Patent Application No. 62/164,955 filed May 21, 2015, the entire contents of which are incorporated by reference herein.

### FIELD OF THE DISCLOSURE

The present invention relates generally to the field of data processing and data structures, and more particularly, to processing and transforming data structures for patient medical data, such as clinical trial data, to facilitate review and analysis of such data.

### BACKGROUND

Clinical trials may be used to study the safety and/or efficacy of drugs, medical devices, medical treatments, procedures, and the like for use in obtaining regulatory approvals from regulatory agencies, such as the FDA. Clinical trials may typically involve several phases and hundreds or thousands of patients (subjects) and may span several years. Reports submitted to regulatory agencies in connection with clinical trials may include, among other things, patient clinical data dispersed among various categories spanning thousands of pages, as well statistical analyses regarding safety, efficacy, adverse events, and the like. The amount of data generated from a single clinical trial is vast, and the data generated for any given patient of the clinical trial is itself voluminous.

Conventional data processing systems and methods have sought to facilitate the collection, management, security, updating and sharing of data for clinical trials as well as the integration of computer systems and database systems that support the administration of clinical trials. Physicians and other decision makers nevertheless remain faced with a vast universe of data for analysis in any given clinical trial.

### SUMMARY

The inventors have observed and appreciated that conventional reporting and presentation of patient data associated with clinical trials remains insufficient to convey to decision makers, particularly physicians, relevant patient data in an efficient and streamlined manner to support decision making. The inventors have observed and appreciated a need in connection with clinical trials for improved data processing and presentation of patient data to facilitate review and analysis of clinical data for any given patient such that physicians may quickly review and understand important data related to the patient's physiology, treatment, testing, adverse effects, and response to treatment without being inundated with less remarkable patient information. Aspects of the present disclosure may address various deficiencies in the conventional art observed by the present inventors.

According to one example, the present disclosure describes a computerized method of transforming patient data formatted according to one or more computer data structures into another format of another computer data structure for efficient processing and visualization of patient data. The method comprises: defining a first data structure comprising multiple data fields in which to store patient data for a plurality of patients; accessing source data structured according to one more other data structures for the plurality of patients, the source data being arranged among plural data fields of the one or more data structures, each of the plural data fields of the one or more other data structures having a data type, each of the plural data fields of the one or more other data structures having a content type, the one or more other data structures being different in structure from the first data structure; selecting desired source data for the plurality of patients from the plural data fields of the one or more other data structures; assigning selected source data from the plural data fields of the one or more other data structures to the multiple data fields of the first data structure to populate the patient data of the first data structure, wherein the assigning includes transforming source data of different content types for two or more fields of the one or more other data structures into a particular transformative data field of the first data structure, wherein the multiple data fields of the first data structure include data fields for multiple treatment and assessment cycles at different times; and processing the patient data of the first data structure for visualization at a graphical user interface such that representations of fields of the multiple data fields of the first data structure for the given patient are arranged for display via a single window of the graphical user interface, and such that certain fields of the multiple fields of the first data structure are structured so as to not visibly convey numerical or textual data via the graphical user interface, wherein at least some of the certain fields convey substantive information by virtue of not displaying visible numerical or textual data. By selecting only certain data from multiple data structures of plural databases and consolidating the selected data into the first data structure, which can be smaller in size than the source data structure(s), the exemplary approaches described herein may provide a technical advantage of efficient processing of data because the data may be accessed and analyzed at a computer more quickly and efficiently and may also provide a technical advantage of reduction in size of storage resources and allocation of computing resources needed for processing, visualizing and analyzing data of the first data structure. Moreover, by making the presentation and visualization of data of the first data structure simpler, physicians and other decisions makers may evaluate the data of the first data structure more quickly, and this may provide a technical advantage of permitting greater resource allocation and bandwidth to other computer applications and processes among networked computers that access the same computer server network.

According to another example, the present disclosure describes a computer system for transforming patient data formatted according to one or more computer data structures into another format of another computer data structure for efficient processing and visualization of patient data. The system comprises a processing system and a memory coupled to the processing system, wherein the system is configured to execute steps comprising: defining a first data structure comprising multiple data fields in which to store patient data for a plurality of patients; accessing source data structured according to one or more other data structures for the plurality of patients, the source data being arranged among plural data fields of the one or more data structures, each of the plural data fields of the one or more other data structures having a data type, each of the plural data fields of the one or more other data structures having a content type, the one or more other data structures being different in structure from the first data structure; selecting desired source data for the plurality of patients from the plural data fields of the one or more other data structures; assigning selected source data from the plural data fields of the one or more other data structures to the multiple data fields of the first data structure to populate the patient data of the first data structure, wherein the assigning includes transforming source data of different content types for two or more fields of the one or more other data structures into a particular transformative data field of the first data structure, wherein the multiple data fields of the first data structure include data fields for multiple treatment and assessment cycles at different times; and processing the patient data of the first data structure for visualization at a graphical user interface such that representations of fields of the multiple data fields of the first data structure for the given patient are arranged for display via a single window of the graphical user interface, and such that certain fields of the multiple fields of the first data structure are structured so as to not visibly convey numerical or textual data via the graphical user interface, wherein at least some of the certain fields convey substantive information by virtue of not displaying visible numerical or textual data. By selecting only certain data from multiple data structures of plural databases and consolidating the selected data into the first data structure, which can be smaller in size than the source data structure(s), the system may provide a technical advantages of efficient processing of data because the data may be accessed and analyzed at a computer more quickly and efficiently and may also provide a technical advantage of reduction in size of storage resources and allocation of computing resources needed for processing, visualizing and analyzing data of the first data structure. Moreover, by making the presentation and visualization of data of the first data structure simpler, physicians and other decisions makers may evaluate the data of the first data structure more quickly, and this may provide a technical advantage of permitting greater resource allocation and bandwidth to other computer applications and processes among networked computers that access the same computer server network.

According to one example, the present disclosure describes a non-transitory computer readable medium for transforming patient data formatted according to one or more computer data structures into another format of another computer data structure for efficient processing and visualization of patient data. The computer readable medium comprises computer instructions which when executed cause a computer processing system to execute steps comprising: defining a first data structure comprising multiple data fields in which to store patient data for a plurality of patients; accessing source data structured according to one or more other data structures for the plurality of patients, the source data being arranged among plural data fields of the one or more data structures, each of the plural data fields of the one or more other data structures having a data type, each of the plural data fields of the one or more other data structures having a content type, the one or more other data structures being different in structure from the first data structure; selecting desired source data for the plurality of patients from the plural data fields of the one or more other data structures; assigning selected source data from the plural data fields of the one or more other data structures to the multiple data fields of the first data structure to populate the patient data of the first data structure, wherein the assigning includes transforming source data of different content types for two or more fields of the one or more other data structures into a particular transformative data field of the first data structure, wherein the multiple data fields of the first data structure include data fields for multiple treatment and assessment cycles at different times; and processing the patient data of the first data structure for visualization at a graphical user interface such that representations of fields of the multiple data fields of the first data structure for the given patient are arranged for display via a single window of the graphical user interface, and such that certain fields of the multiple fields of the first data structure are structured so as to not visibly convey numerical or textual data via the graphical user interface, wherein at least some of the certain fields convey substantive information by virtue of not displaying visible numerical or textual data. By selecting only certain data from multiple data structures of plural databases and consolidating the selected data into the first data structure, which can be smaller in size than the source data structure(s), the approach may provide a technical advantage of efficient processing of data because the data may be accessed and analyzed at a computer more quickly and efficiently and may also provide a technical advantage of reduction in size of storage resources and allocation of computing resources needed for processing, visualizing and analyzing data of the first data structure. Moreover, by making the presentation and visualization of data of the first data structure simpler, physicians and other decisions makers may evaluate the data of the first data structure more quickly, and this may provide a technical advantage of permitting greater resource allocation and bandwidth to other computer applications and processes among networked computers that access the same computer server network.

According to an example, the present disclosure describes a computer data structure for facilitating efficient processing and visualization of patient data. The data structure comprises: multiple data fields in which to store patient data for a plurality of patients, the multiple data fields of the data structure including a first set of data fields for storing screening information for the given patient, a second set of data fields for storing treatment information for the given patient, a third set of data fields for storing information regarding patient response to treatment for the given patient, a fourth set of data fields for storing laboratory results information for the given patient, a fifth set of data fields for storing medication information for the given patient, and a sixth set of data fields for storing adverse event information for the given patient, wherein the second, third, fourth, fifth and sixth sets of data fields include data fields for multiple treatment and assessment cycles, wherein some data fields of the multiple data fields are configured to store patient data of two or more content types in a single data field, and wherein the second, third, fourth, fifth and sixth sets of data fields include data fields for multiple treatment cycles, and wherein certain fields of the multiple fields of the first data structure are structured so as to not visibly convey numerical or textual data via the graphical user interface, wherein at least some of the certain fields convey substantive information by virtue of not displaying visible numerical or textual data. The first data structure can be smaller in size than the source data structure(s), being comprised of only certain selected data from multiple source data structures of plural databases and consolidated into, for example, one first data structure, and this may provide a technical advantage of efficient processing of data because the data may be accessed and analyzed at a computer more quickly and efficiently. The first data structure may also provide a technical advantage in size reduction of storage resources and a reduction in allocation of computer resources needed for processing, visualizing and analyzing data of the first data structure. Moreover, by making the presentation and visualization of data of the first data structure simpler, physicians and other decisions makers may evaluate the data of the first data structure more quickly, and this may provide a technical advantage of permitting greater resource allocation and bandwidth to other computer applications and processes among networked computers that access the same computer server network.

### DETAILED DESCRIPTION OF THE DRAWINGS

Exemplary embodiments will now be described with reference to the accompanying drawings.

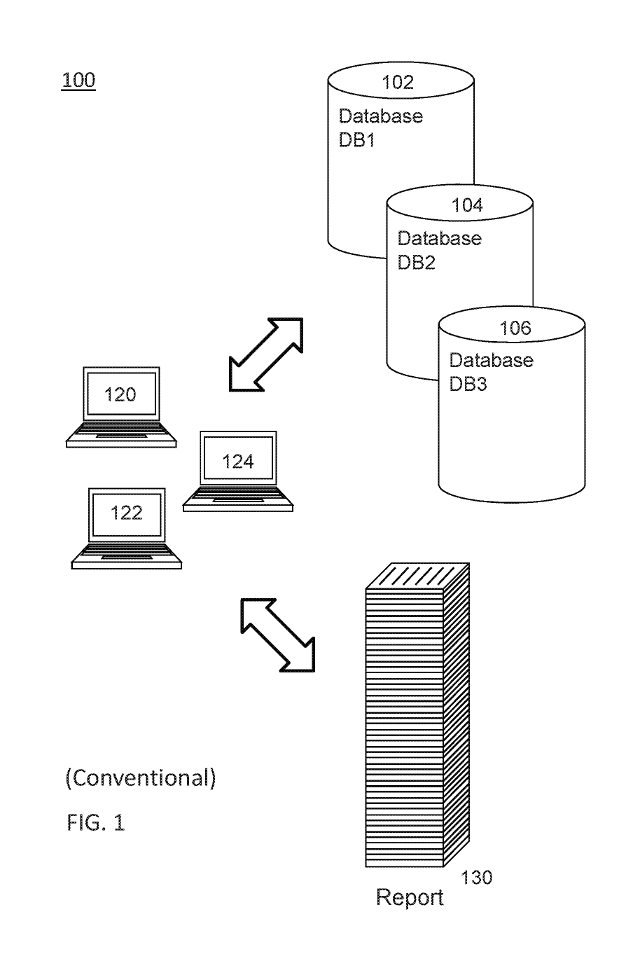

FIG. 1 illustrates a conventional framework for analyzing and reporting patient data in clinical trials.

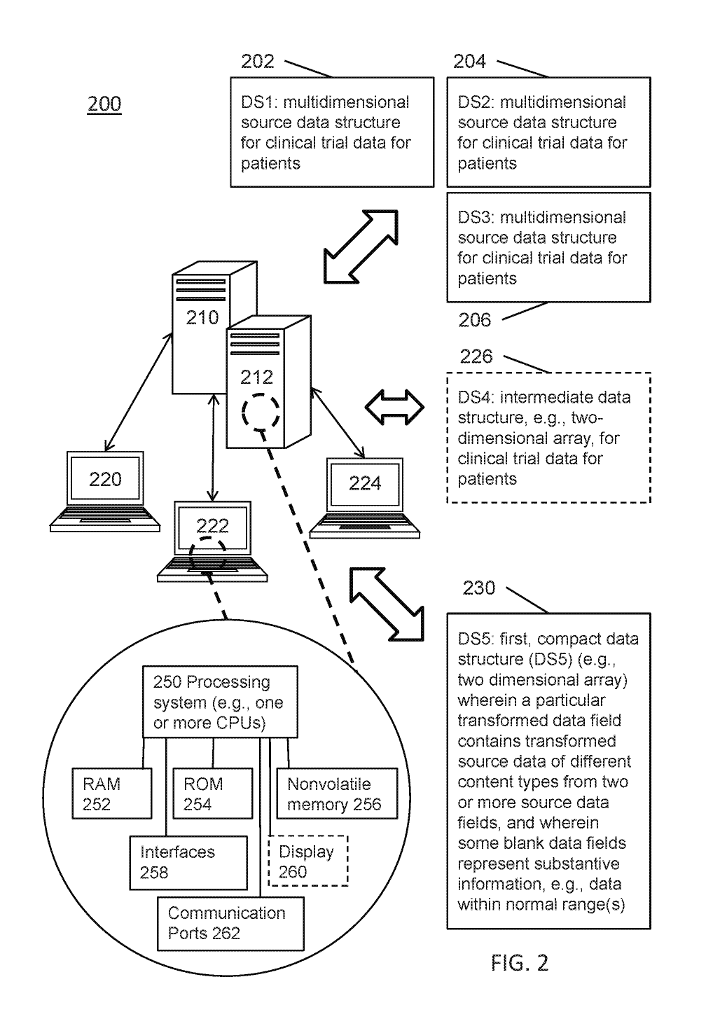

FIG. 2 illustrates an exemplary system and approach for transforming source patient data formatted according to one or more computer data structures into a different format of a different, e.g., compact computer data structure according to an aspect of the present disclosure.

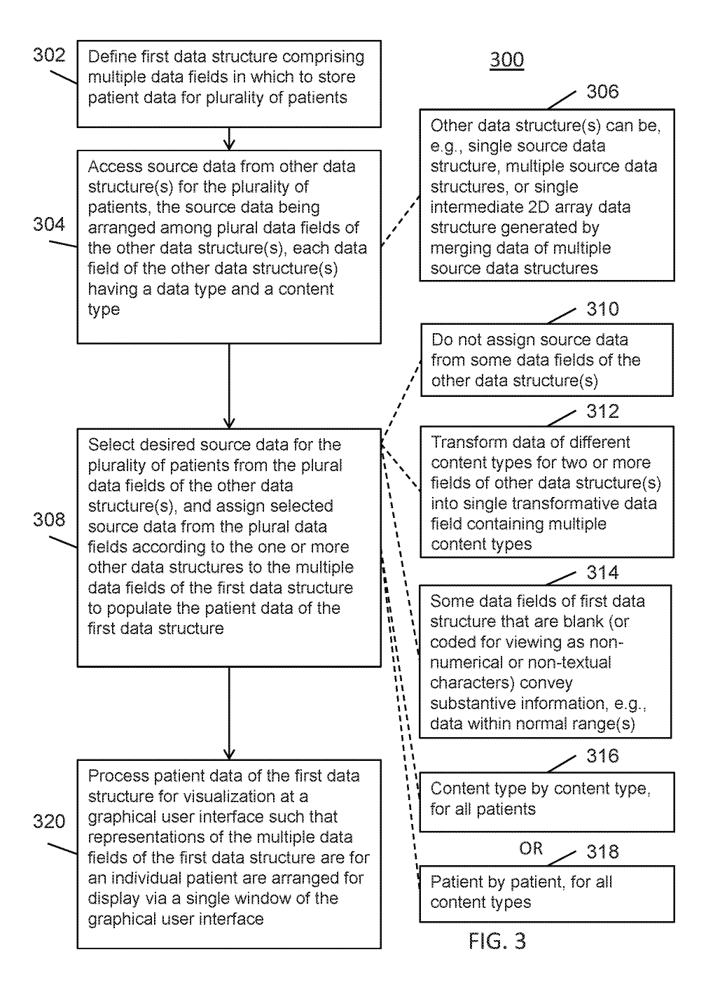

FIG. 3 illustrates a flow diagram for an exemplary method of transforming source patient data formatted according to one or more computer data structures into different format of a different, e.g., compact computer data structure according to an aspect of the present disclosure.

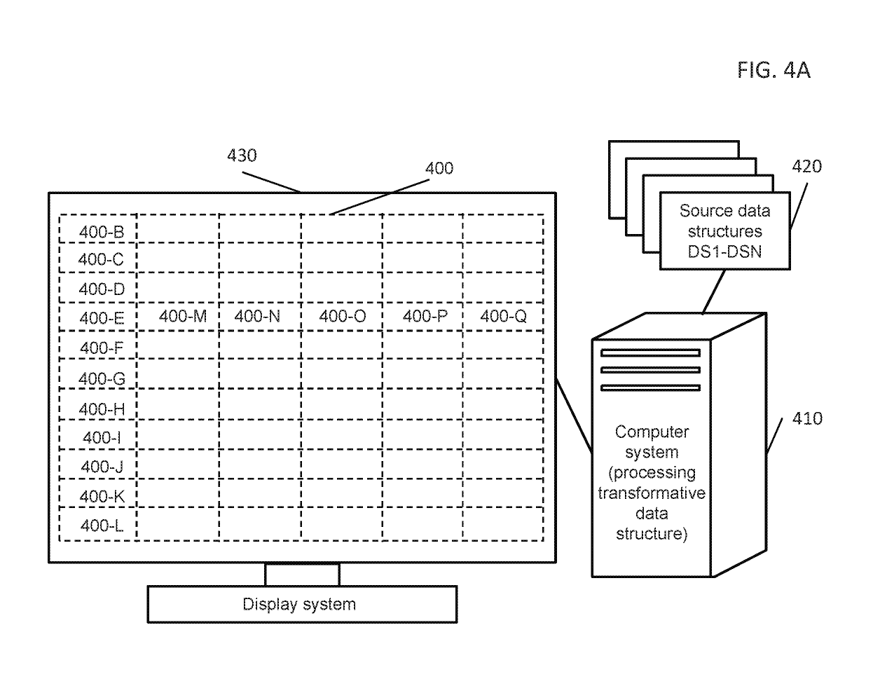

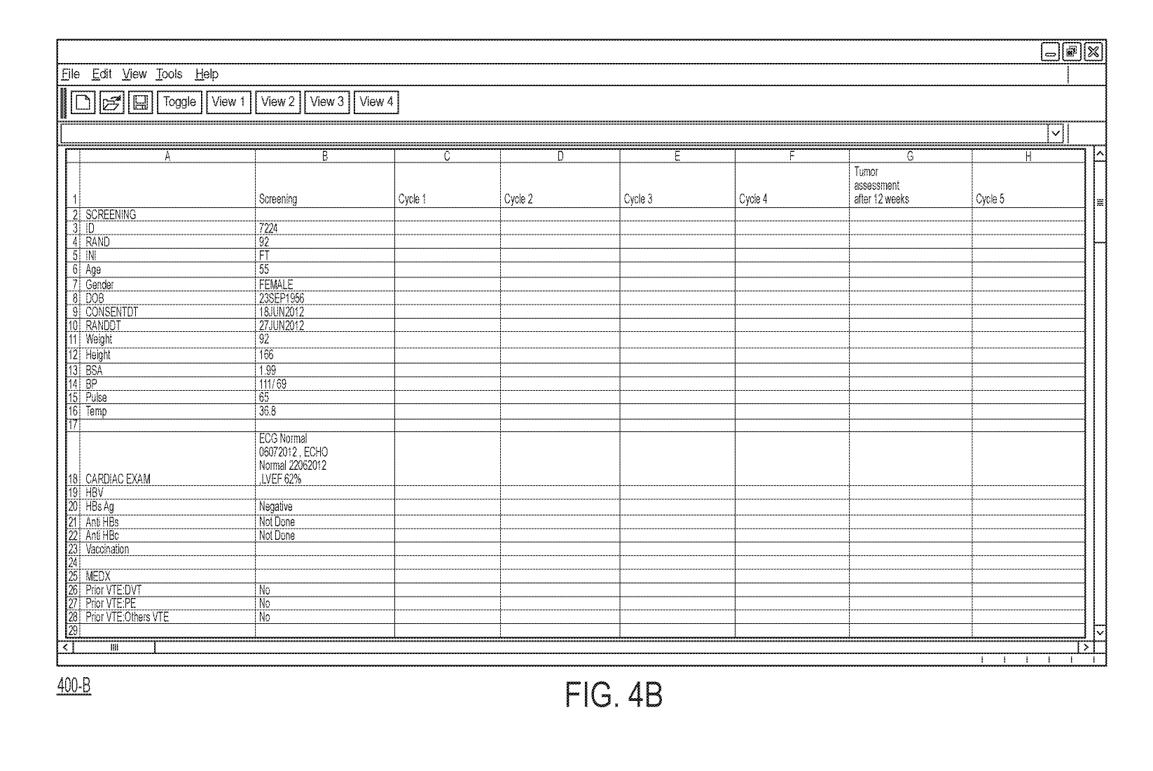

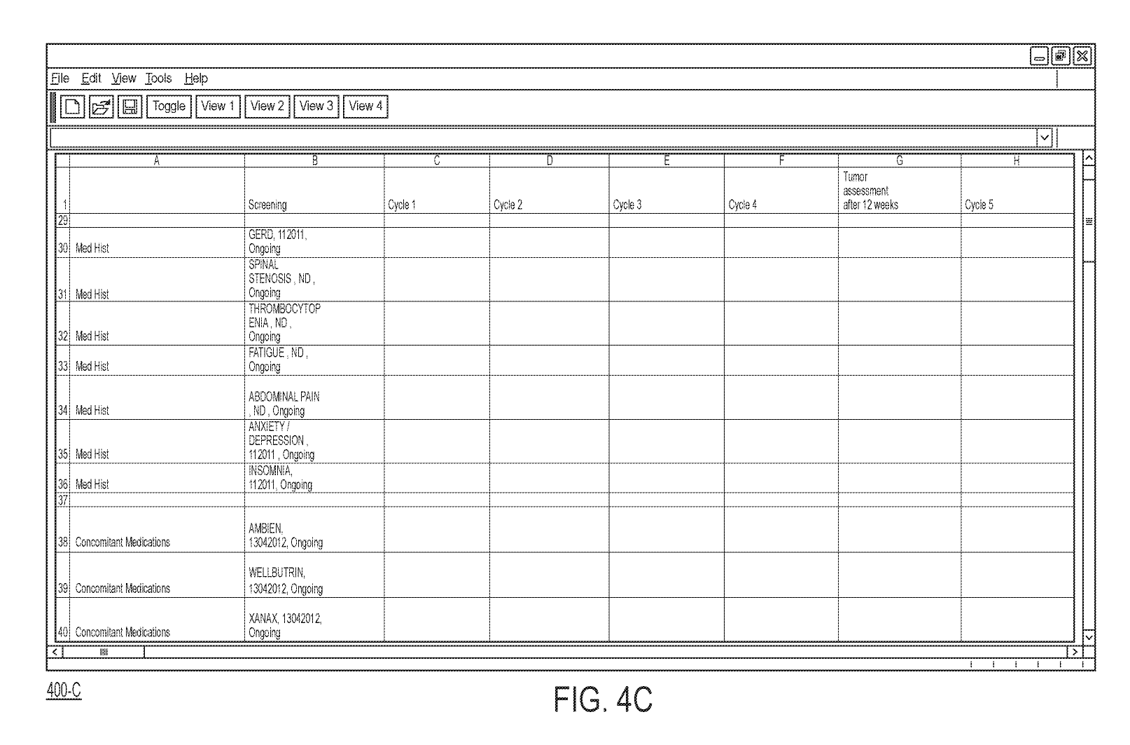

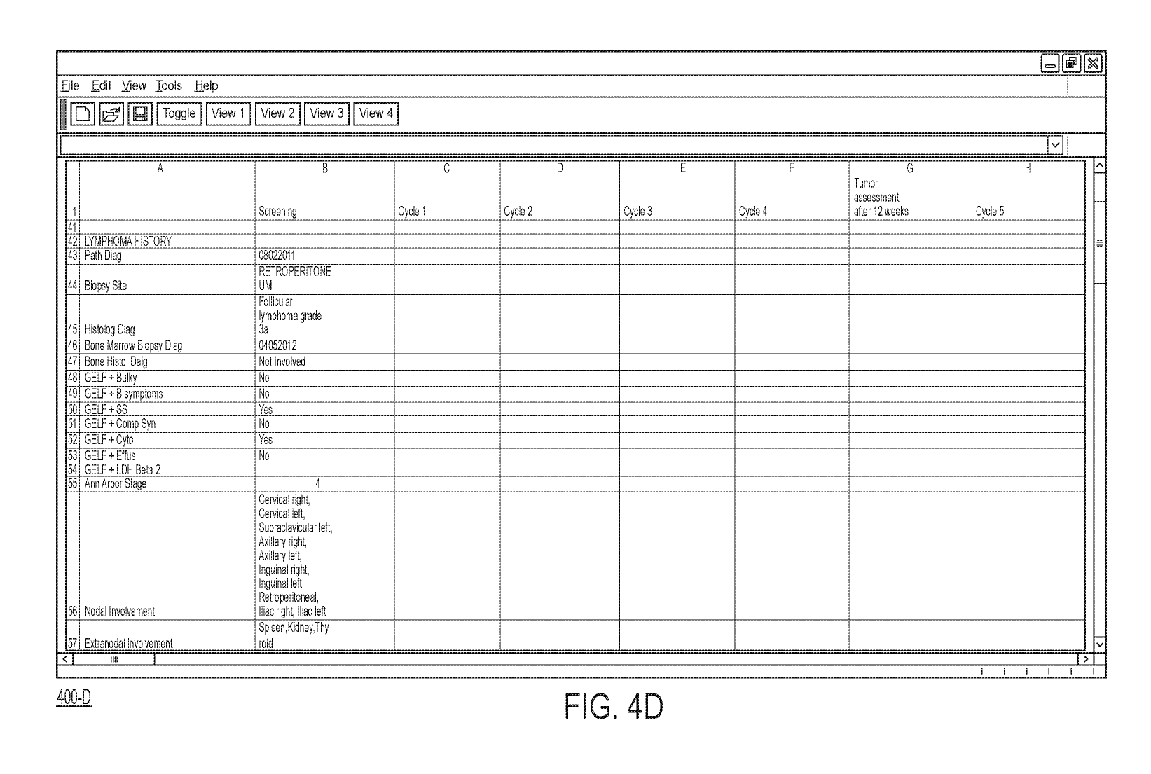

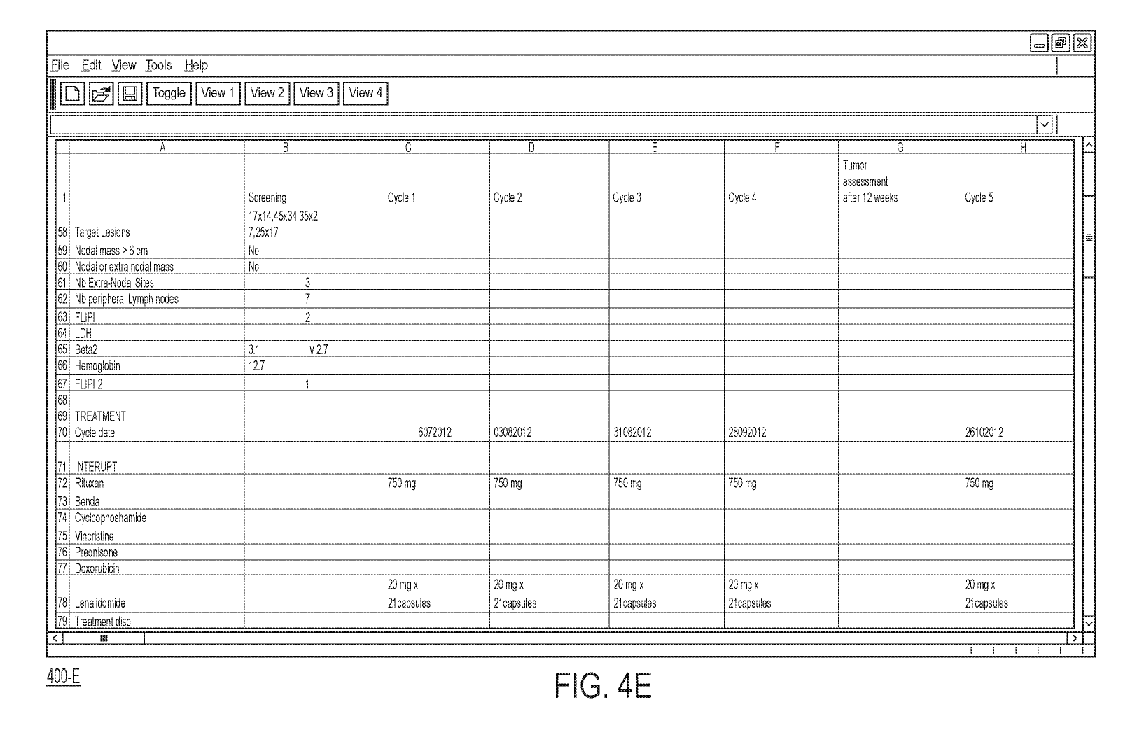

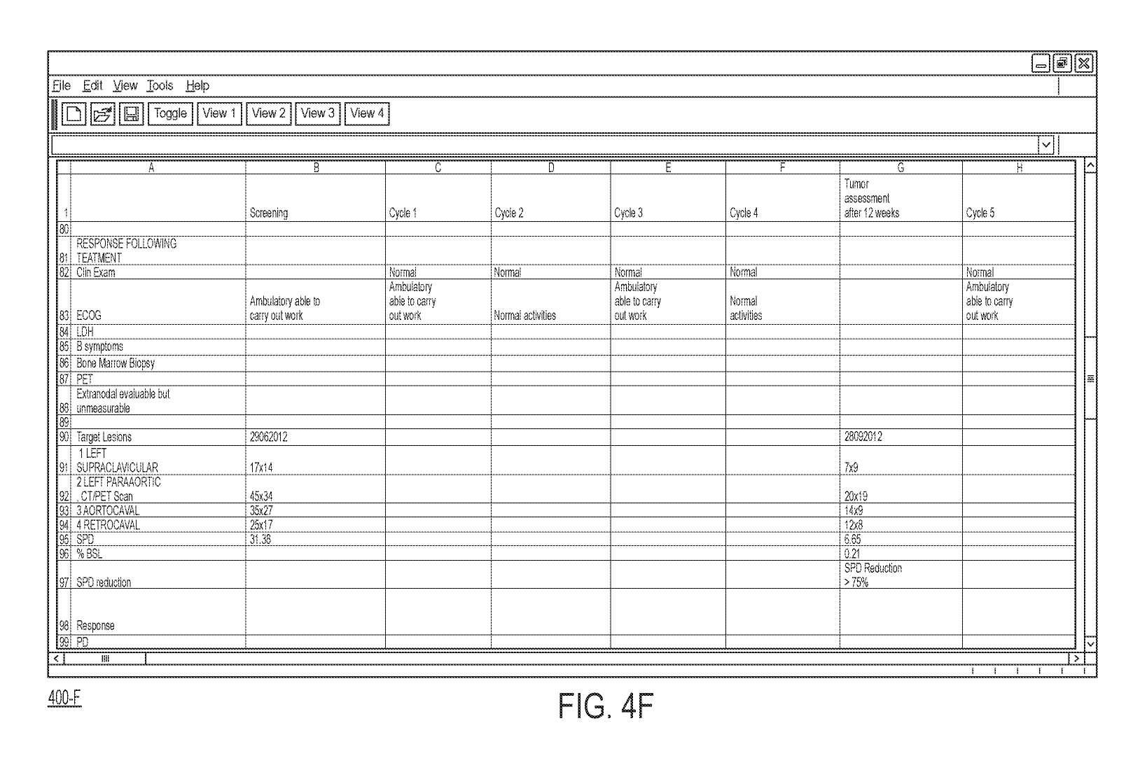

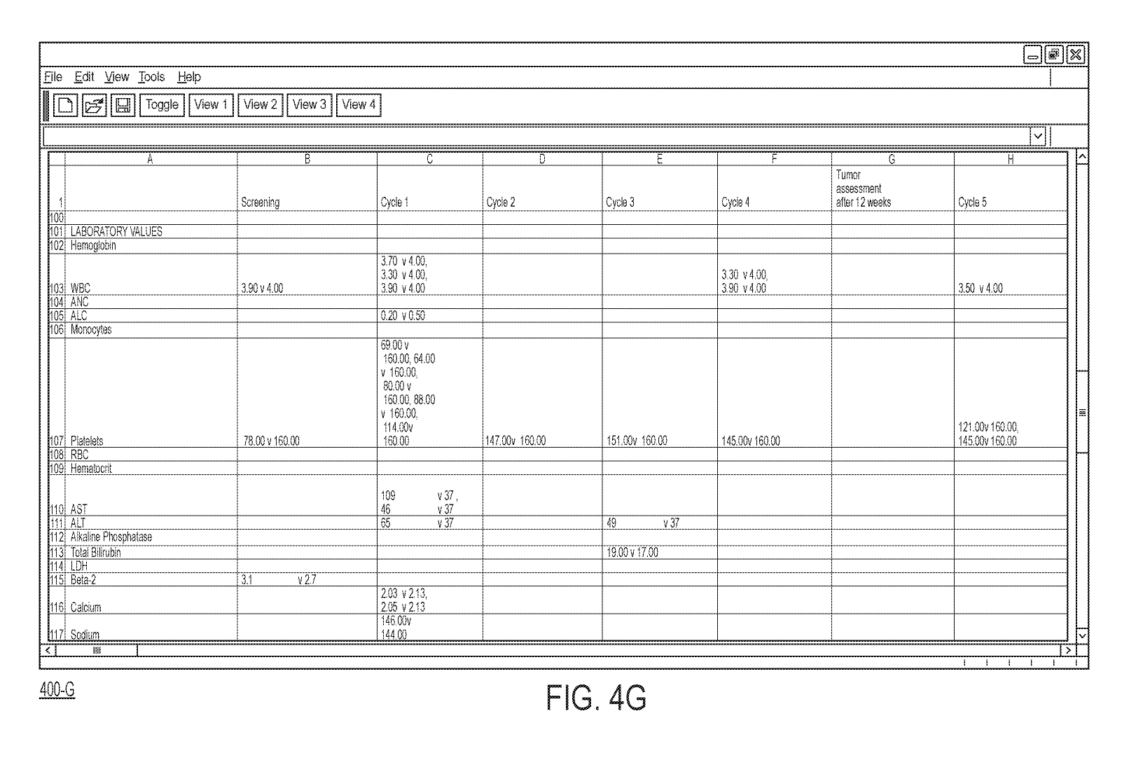

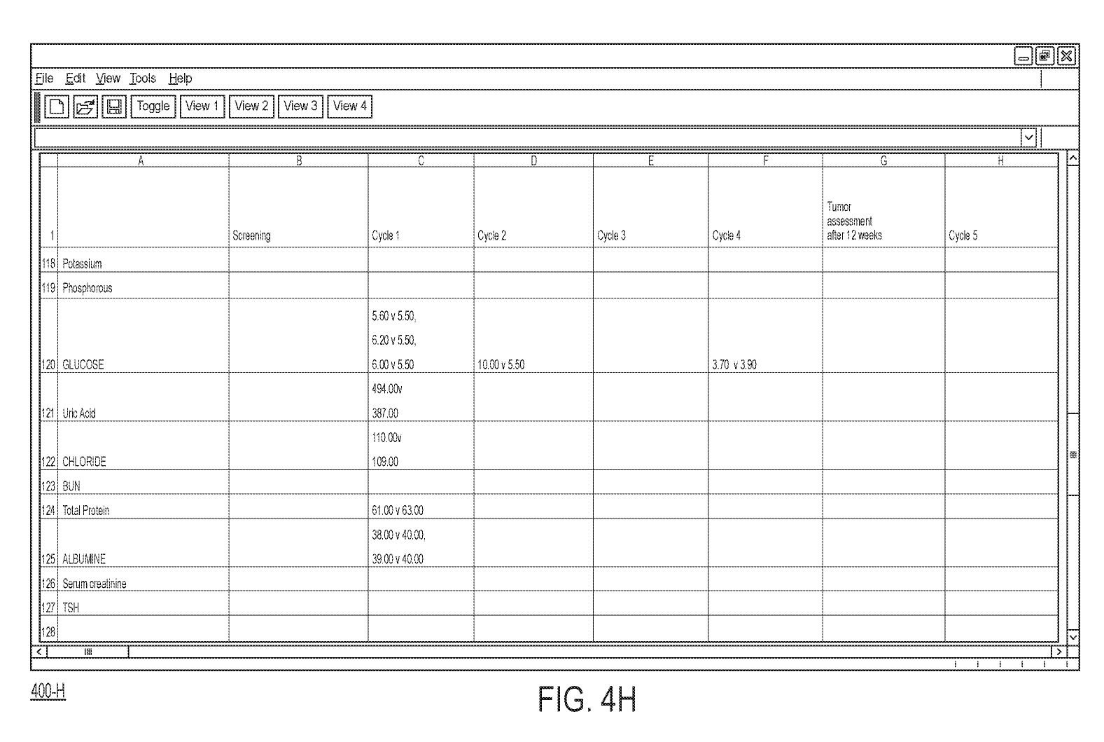

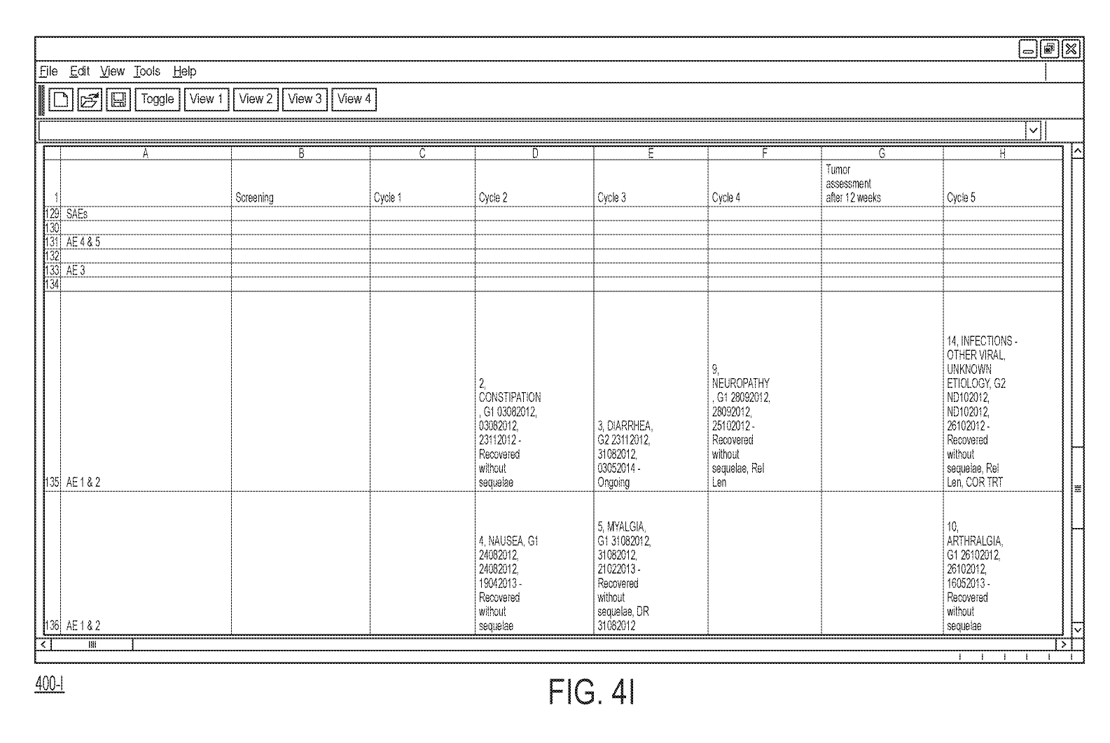

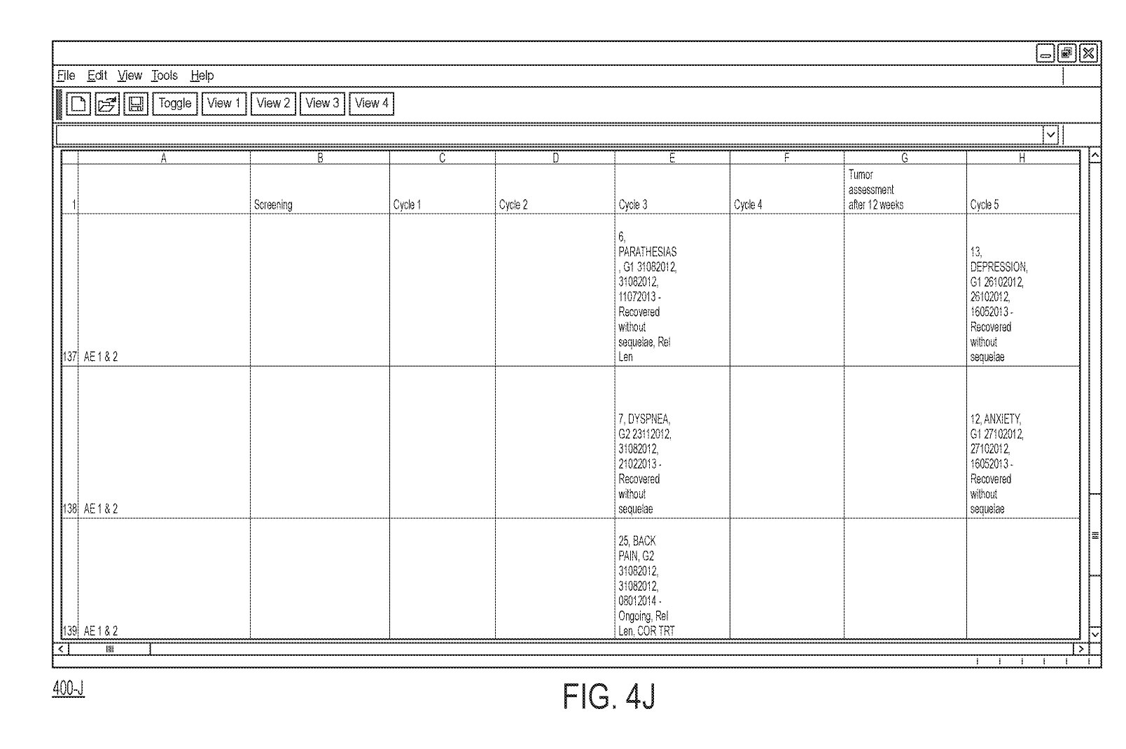

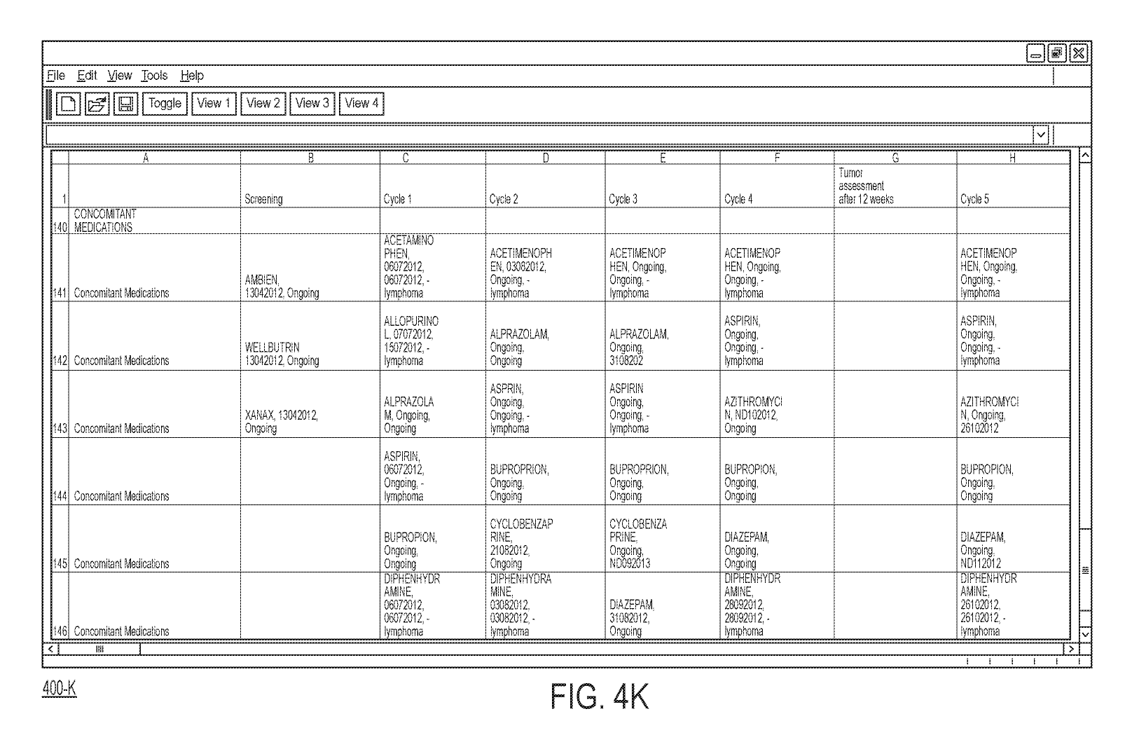

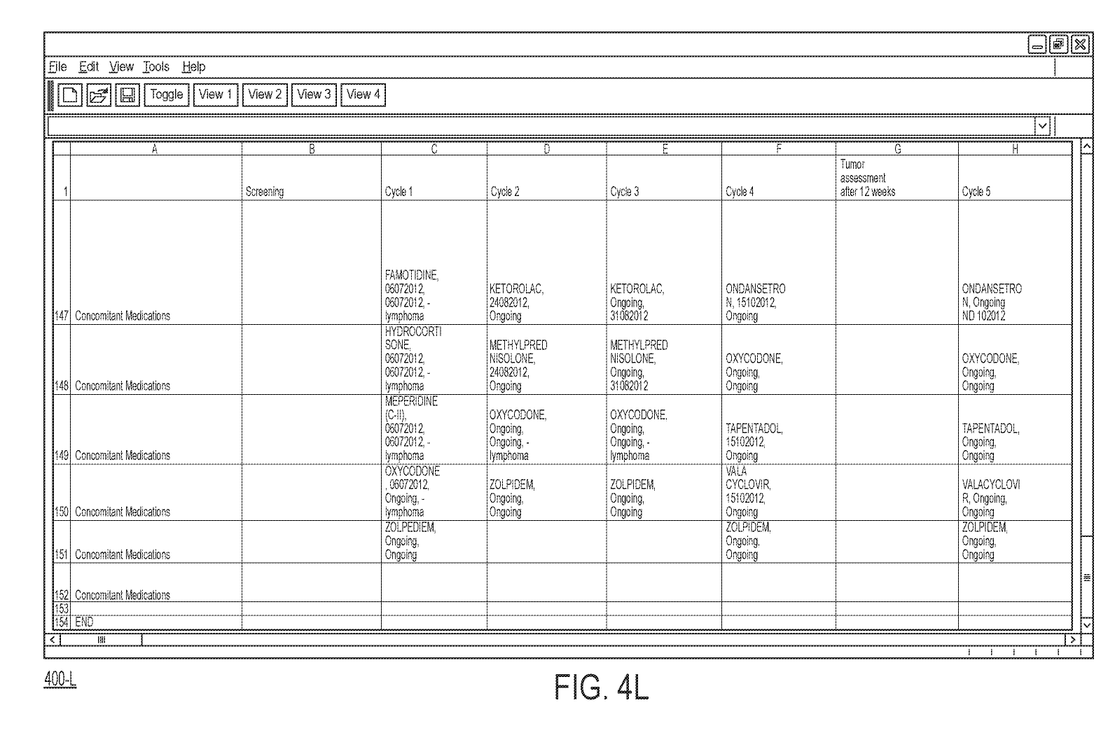

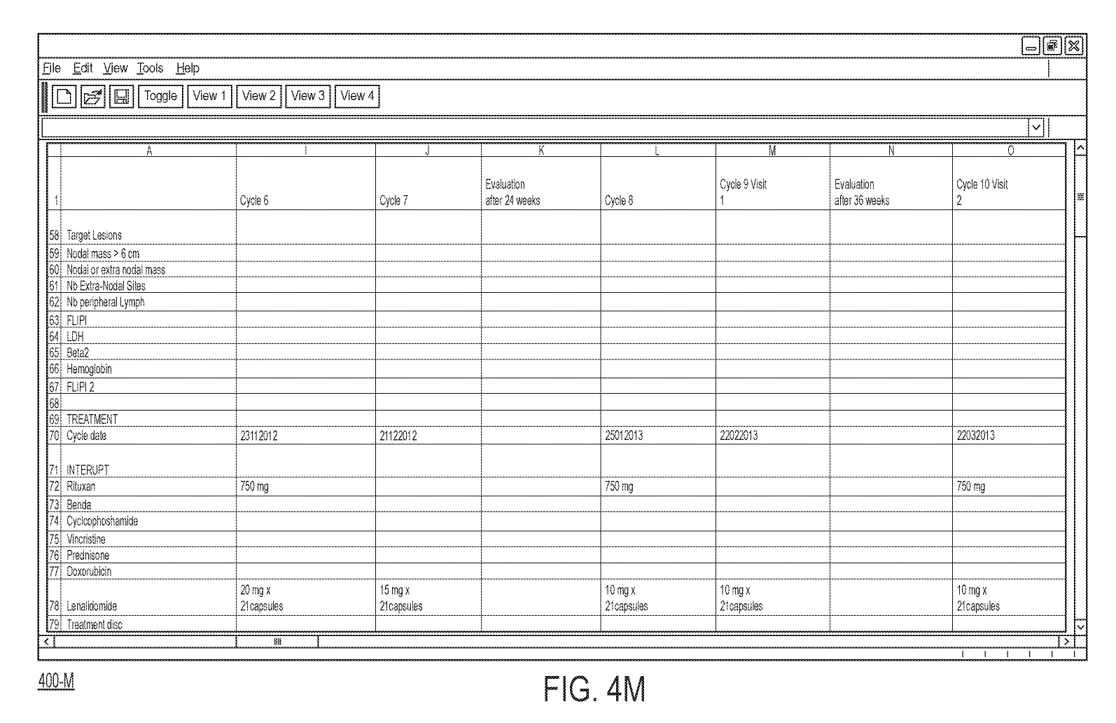

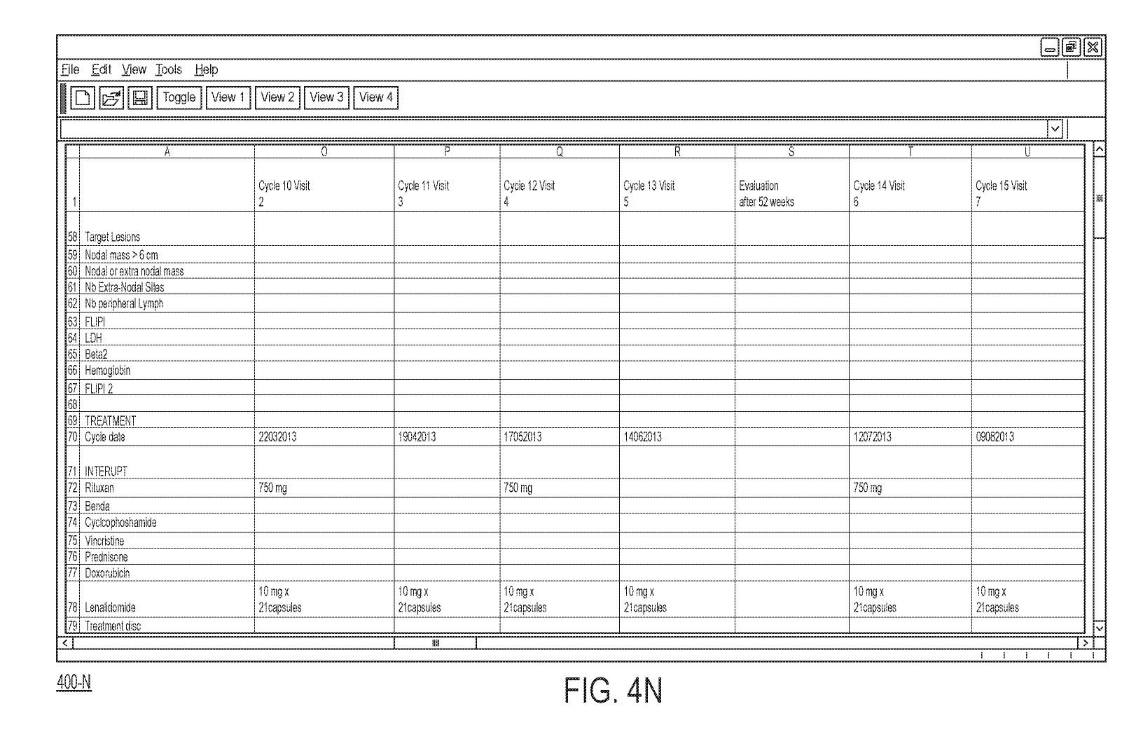

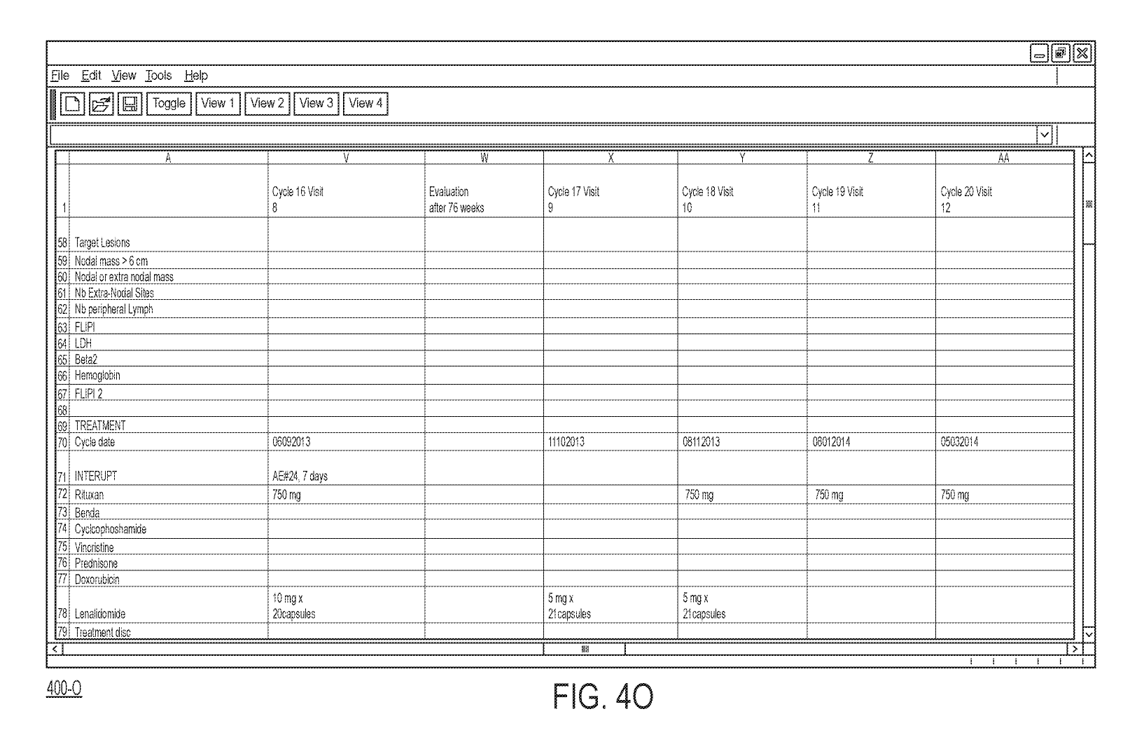

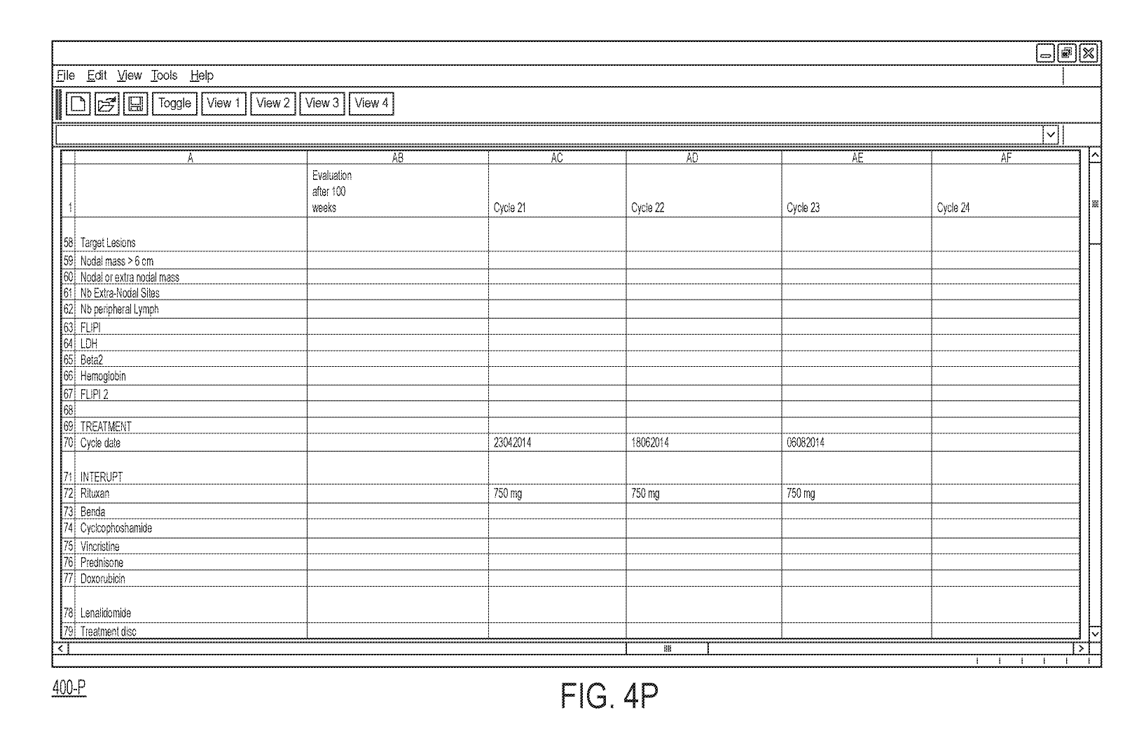

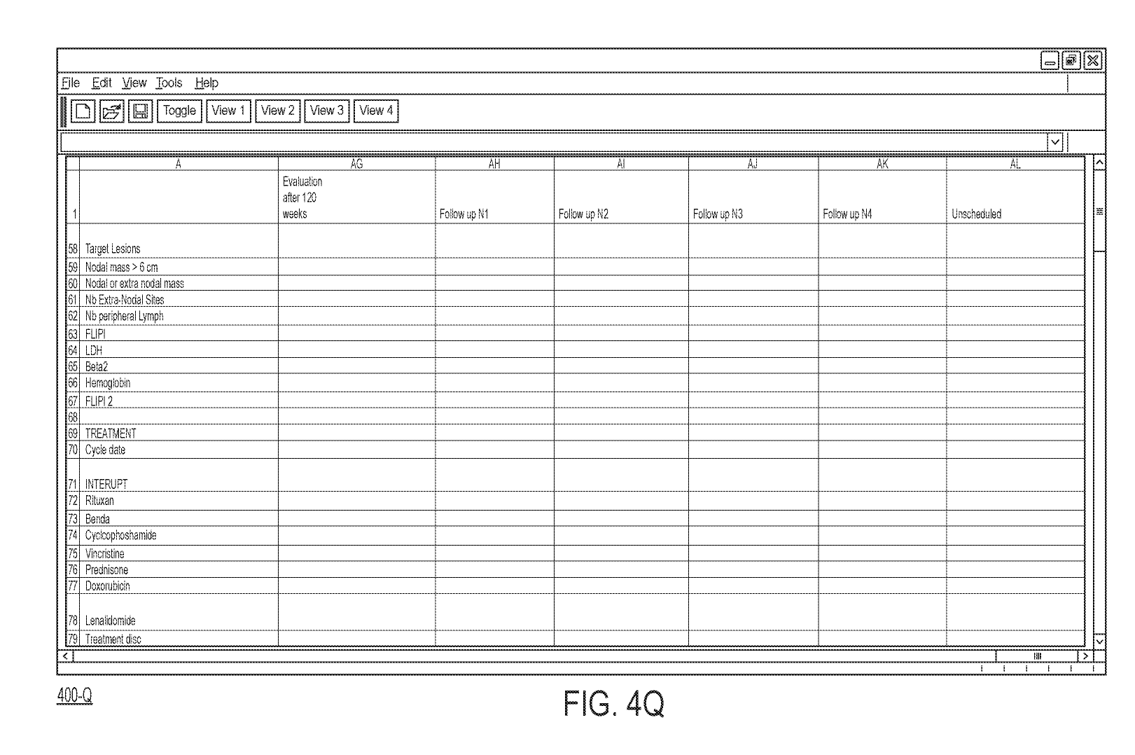

FIG. 4A illustrates an exemplary system for viewing patient data via a graphical user interface according to an aspect of the disclosure.

FIGS. 4B-4Q illustrate portions of an exemplary page of a graphical user interface that presents in two-dimensions transformed patient data according to an aspect of the disclosure.

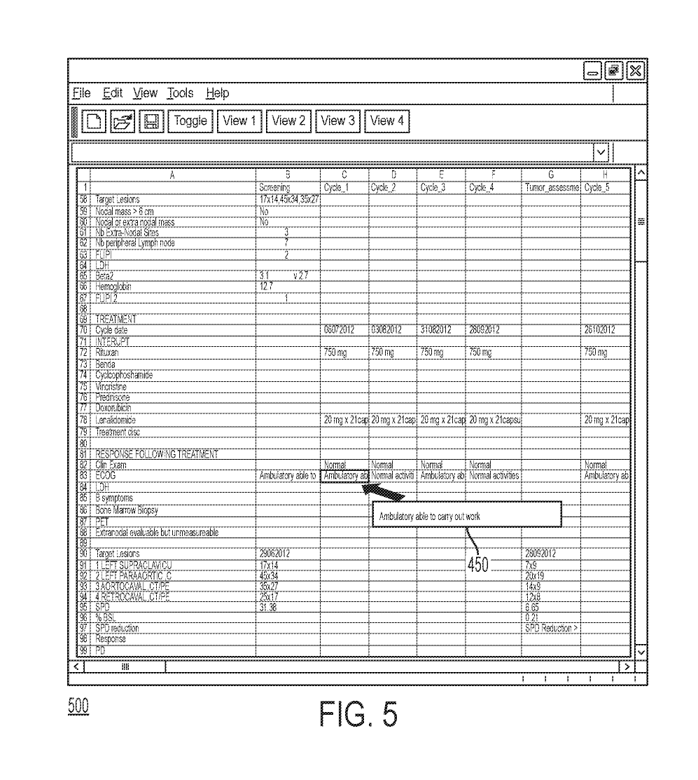

FIG. 5 illustrates another exemplary page of a graphical user interface for viewing patient data according to an aspect of the disclosure.

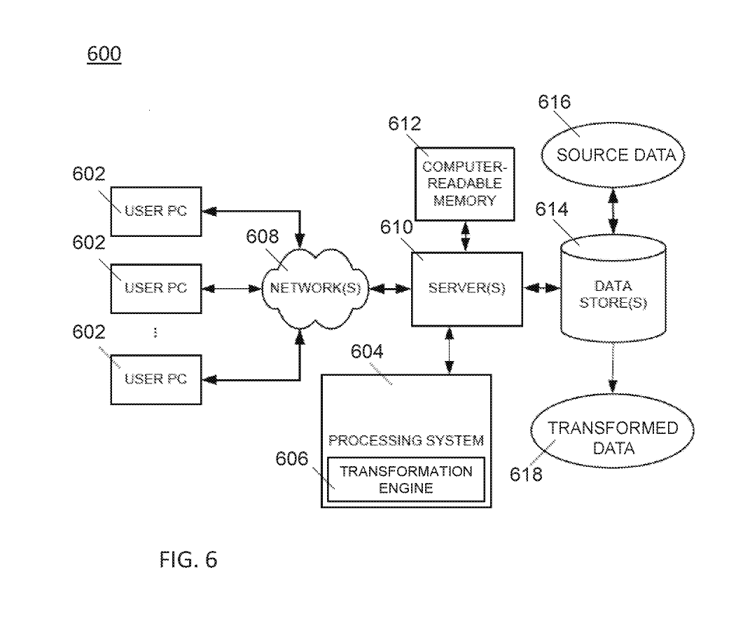

FIG. 6 illustrates a block diagram of an exemplary computer system for use in transforming source patient data formatted according to one or more computer data structures into different format of a different, e.g., compact computer data structure according to an to an aspect of the present disclosure.

### DETAILED DESCRIPTION OF EXEMPLARY EMBODIMENTS

Exemplary aspects of the present disclosure are directed at solutions for transforming source data formatted according to one or more source data structures for multiple patients engaged in treatment, e.g., clinical trials, into transformed data formatted according to a first, e.g., compact, data structure so that the transformed data can be easily and efficiently presented to physicians and other decision makers via a graphical user interface (GUI) that conveys important patient information for a given patient on a single page of the GUI. Files containing transformed data according to the first data structure can be substantially smaller in size (e.g., storage size in megabytes, gigabytes, etc.) than files containing source data according to the one or more source data structures. Transformed data formatted according to the first data structure may facilitate review, comprehension and subsequent processing of important data related to a patient's physiology, treatment, testing, and response to treatment by a physician or other decision maker without being inundated with less remarkable patient information.

FIG. 1 illustrates a conventional framework 100 for analyzing and reporting patient data in clinical trials. The framework 100 includes source databases 102 (DB1), 104 (DB2) and 106 (DB3), which contain patient information for a plurality of patients engaged in one or more clinical trials. This patient information is typically vast and may include information about the patients' physical attributes, medical history, treatments being undertaken, response to treatments, adverse effects, and the like. Information relating to the patients' treatment, response to treatment, and adverse effects is typically collected over a period of time that may span months to years.

The framework 100 shown in FIG. 1 includes user computers 120, 122, and 124 in communication with the databases 102, 104, 106. The user computers 120, 122, 124 may be computer workstations, e.g., PCs, that permit physicians, technicians, or other authorized personnel to access the patient information in databases 102, 104, 106 for analysis and processing so as to be able to prepare a report 130 that may be used, e.g., in a regulatory submission for approval of a product in connection with the clinical trial(s). Typically, the report 130 is itself voluminous and may include thousands of pages. The inventors have observed and appreciated that conventional reporting in connection with clinical trials may inundate and overwhelm physicians and other decision-makers with vast amounts of data in a way that impedes the physicians' and decision-makers' ability to efficiently analyze and comprehend such data. The present inventors have observed that the conventional framework 100 exhibits technical problems of requiring large storage resources, requiring large calculation resources, and requiring high volumes of data to be transferred and processed. The exemplary approaches, systems and data structures described herein may address these technical problems by reducing the amount of data to be processed and reducing a need for ongoing communication with multiple data databases and ongoing access to multiple data structures. The present inventors have also observed and appreciated a need in connection with clinical trials for improved data processing and presentation of patient data to facilitate review, analysis and subsequent processing of clinical data for any given patient such that physicians may quickly review and understand important data related to the patient's physiology, treatment, testing, adverse effects and response to treatment without being inundated with less important patient information. The exemplary approaches, systems and data structures described herein may address these technical problems by making the presentation and visualization of data of the first data structure simpler, such that physicians and other decisions makers may evaluate the data of the first data structure more quickly, and this may provide a technical advantage of permitting greater resource allocation and bandwidth to other computer applications and processes among networked computers that access the same computer server network.

FIG. 2 illustrates an exemplary system 200 and approach for transforming source patient data formatted according to one or more computer data structures into a different format of a different, e.g., compact, computer data structure according to an aspect of the present disclosure. It will be appreciated that a computer file configured with particular data fields in a particular arrangement, which may or may not contain substantive data, may be considered a data structure. The system 200 includes one or more user computer systems 220, 222, 224, which may communicate with server computers 210, 212, so as to access and process patient data for a plurality of patients contained in data structures 202, 204 and 206, also labeled data structures DS1, DS2, and DS3, respectively, in connection with one or more clinical trials. One or more of data structures 202, 204, 206 may be multidimensional data structures in the sense that they may index data and relate data using more than 2 dimensions. For example, a typical relational database may structure data in two dimensions, e.g., such as for rows and columns of a spreadsheet, whereas a multidimensional data structure may structure data for organization in three dimensions, such as a data cube, or more dimensions, such as a hyper-cube. As shown in FIG. 2, a user computer 220, 222, 224, as well as a server computer 210, 212, may each include a processing system 250, which may be comprised of one or more CPUs (central processing unit), various non-transitory memory such as random access memory (RAM) 252, read only memory (ROM) 254, and nonvolatile memory 256 (e.g., hard disk, flash memory, optical disk, etc.), communication ports 262, display 260 (e.g., for user computer work stations though possibly not for server computers), and various interfaces 258 such as user interfaces (e.g., mouse, keyboard), other communications interfaces, and display interfaces, etc. The data structures 202, 204, 206 may be spread across multiple different databases.

In addition, each of the data structures 202, 204, 206 contain data stored in individual data fields thereof. Each data field may have a particular data type (e.g., integer, date, character, etc.), and contain data of a single, particular substantive content type (e.g., patient identification number, age, gender, height, weight, blood pressure, pre-existing medical condition, concomitant medication, treatment medication administered, dosage, date of dosing, adverse effect, laboratory test identification, laboratory test result, date of test result, physical test identification, physical test result, date of physical test, tumor measurement, date of tumor measurement, etc., to name a few). The particular content types of information recorded in data structures 202, 204, 206 will depend upon the particular clinical trial at hand, e.g., treatment for cancer, liver disease, mental illness, or other disease, medical device testing, etc., as will be appreciated by those of ordinary skill in the art. The fact that source data structures 202, 204, 206, may be configured such that each data field thereof contains data of only a single content type may permit maximum flexibility and predictability in manipulating and analyzing the patient data stored in those files according to those data structures, which can reduce the complexity and increase the speed of processing data in the source data structures when generating the first data structure 230.

Any or all of the computer systems 220, 222, 224 may process patient data for multiple patients for a clinical trial (patient data may also be referred to as clinical trial data) stored in data structures 202, 204, 206 so as to merge the patient data and save it to an intermediate data structure 226, also labeled DS4, configured as a two dimensional array. The intermediate data structure 226 may be relatively large in size because it contains clinical trial data for potentially hundreds or thousands of patients or more spanning treatment cycles over a period ranging from months to years. Such an intermediate data structure 226 configured as a two-dimensional array may not itself be suitable for physicians or other decision makers to use for directly visualizing the clinical trial data for any given patient, e.g., in a single two dimensional spreadsheet form, due to the enormity of its content and unwieldy size for direct visualization. In this regard, generating an intermediate data structure 226 configured as a single two dimensional array as described would be viewed as contrary to the conventional wisdom by one of ordinary skill in the art. However, the present inventors have observed that such an intermediate data structure configured as a single two-dimensional array may be beneficial for enhanced processing simplicity for generating a first data structure 230 as explained below. For example, the use of a single intermediate data structure 226 as described in examples herein can provide a technical advantage by reducing the communications resources and bandwidth that would otherwise be needed to communicate on an ongoing basis with a plurality of different databases and to access on an ongoing basis multiple source data structures 202, 204 and 206 potentially for which the plural databases and multiple data structures may be configured and stored according to a variety of different protocols. In other words, exemplary approaches described herein can provide a technical advantage by reducing the number of databases with which to communicate, reducing the number of source data structures to access, and reducing the number of data protocols that need to be managed, thereby increasing speed of access and processing.

Referring again to FIG. 2, any or all of the computer systems 220, 222, 224 may process the patient data of intermediate data structure 226 and save certain desired patient data from intermediate data structure 226 to the first data structure 230, which likewise may be configured as a two dimensional array. However, the use of an intermediate data structure 226, while beneficial, is not required and is optional. For example, source data for multiple patients from data structures 202, 204, 206 (e.g., computer files) may be processed by computer systems 220, 222, and/or to 224 so as to yield transformed data that is directly stored in first data structure 230. In this example, data structure 202 might contain adverse event information for multiple patients, data structure 204 might contain laboratory results, treatment information, and information on response to treatment for the multiple patients, and data structure 206 might contain medical history information for the multiple patients, for instance, and other data structures may contain additional patient information.

As will be described in greater detail elsewhere herein, the first data structure 230 may contain an array of data fields in multiple categories, e.g., a first set of data fields for storing screening information for the given patient, a second set of data fields for storing treatment information for the given patient, a third set of data fields for storing information regarding patient response to treatment for the given patient, a fourth set of data fields for storing laboratory results information for the given patient, a fifth set of data fields for storing medication information for the given patient, a sixth set of data fields for storing adverse event information for the given patient, etc. As shown in FIG. 2, the first data structure 230 may also contain one or more transformative data fields. A transformative data field in this regard contains transformed source data of different content types from two or more source data fields. In other words, where the source patient data for the clinical trial contained two or more pieces of data of different content types, each in a separate data field, the first data structure 230 may contain in one, singular data field transformed data derived from those two or more pieces of data from the source databases. Use of transformative data fields may provide a further technical advantage of reducing the number of data fields in the first data structure and may thereby provide additional size reduction. Insofar as the use of an intermediate data structure 226 is optional as noted above, a source data field in this regard can be either a data field from source data structures 202, 204, 206, from which patient data is obtained, or it can be a source data field of intermediate data structure 226, from which patient data may be obtained.

In addition to having one or more transformative data fields, as shown in FIG. 2, the first data structure 230 may also contain one or more blank data fields that nonetheless represent substantive information by virtue of being blank, e.g., represent substantive patient data values within a normal range or ranges or information that is otherwise unremarkable. In this regard, it can be desirable for certain fields of the first data structure to be structured or coded such that the presentation of corresponding fields or cells at a graphical user interface does not visibly convey or present numerical or textual data, wherein at least some of those certain fields or cells convey substantive information by virtue not displaying or presenting visible numerical or textual data. A blank field can satisfy this requirement. In addition, a dot character, a hyphen character, and other non-numerical and non-textual characters (e.g., other punctuation-type characters) may be coded for certain fields to convey substantive information, e.g., substantive patient information, without presenting the actual numerical or textual data thereof in corresponding fields or cells of the graphical user interface. Non-numerical and non-textual characters include characters that are not alphabetic characters, not numbers, and not otherwise symbolic of substantive content (e.g., not kanzi, kanji, or hanja characters) whether in English or other languages. As will be discussed in greater detail below, this aspect is contrary to the conventional wisdom which indicates that all measurement data values should be presented. The utilization of transformative data fields in the first data structure 230, as well as blank data fields (or data fields with unobtrusive non-numerical or non-textual characters) to represent substantive information, provide substantial reductions in the amount of numerical and character data that reside in first data structure 230, thereby providing substantial improvements and efficiencies to physicians and other decision-makers in reviewing and comprehending clinical trial data for any given patient, as will be discussed in greater detail below. The use of transformative data fields may also provide a technical advantage of reduction in a number of fields in the first data structure and a reduction in size. As will be described below, such blank data fields or data fields with unobtrusive non-numerical or non-textual characters, which may nonetheless convey substantive information, can provide for ease of viewing and comprehending the most salient data with less remarkable data being replaced by white space (e.g., blank fields or fields containing unobtrusive non-numerical and non-textual characters). This can permit displaying at the GUI the appearance of blank data cells or unobtrusive non-numerical or non-textual characters at cells of the GUI, in which case the GUI can likewise be presented in a more compact fashion, and this can provide a technical advantage of reducing the display resources that might otherwise be required, e.g., reducing or eliminating a need for multiple display monitors, multiple video cards, or more sophisticated video cards, and the like.

FIG. 3 illustrates a flow diagram for an exemplary method 300 of transforming source patient data formatted according to one or more source computer data structures into a different format of a different computer data structure (which may be referred to as first data structure, or a first compact data structure herein, e.g., 230) according to an aspect of the present disclosure. The method 300 can be carried out by a computer processing system (e.g., one or more computer processing units or CPUs located in one computer or distributed among multiple computers), such as processing system 250 described in connection with FIG. 2, or with a computer processing system such as will be described herein with regard to FIG. 6. As shown in FIG. 3, at step 302, a first data structure is defined comprising multiple data fields in which to store patient data for a plurality of patients associated with the clinical trial. The first data structure in this regard can be, for example, the first data structure 230 described previously in connection with FIG. 2. These data fields may comprise an array of data fields categorized into various sets or categories, e.g., a first set of data fields for storing screening information for the given patient, a second set of data fields for storing treatment information for the given patient, a third set of data fields for storing information regarding patient response to treatment for the given patient, a fourth set of data fields for storing laboratory results information for the given patient, a fifth set of data fields for storing medication information for the given patient, a sixth set of data fields for storing adverse event information for the given patient, etc. The data fields of any given category may further be grouped together according to the data structure so that data fields in any given category are coded for proximity, e.g., adjacency, to one another in terms field or cell identification to facilitate subsequent data manipulation and viewing, e.g., via a two dimensional page of a spreadsheet of a graphical user interface. An example in this regard will be described further with reference to FIG. 4 herein.

The defining of the first data structure at step 302 may comprise the execution of suitable instructions by the processing system to assign names or identifications to fields of the first data structure, e.g., a data structure configured as a two dimensional array. For example, the defining may assign names or identifications to a two-dimensional array of rows and columns such as for a spreadsheet. Where a two dimensional array is used to represent data for any given patient, the first data structure, e.g., 230, may be configured to include a plurality of such two-dimensional arrays, one for each patient of the plurality of patients. The names and identifications of various fields (e.g., row and column headings) of the first data structure may be chosen by a database engineer, system analyst, physician, or other authorized person. Accordingly, the act of defining at step 302 can be considered to be carried out by the processing system, an authorized person, or both. The defining of the first data structure may also involve technical aspects of specifying the size of the first data structure, the number of data fields of the data structure, data types of the data fields, and the like.

At step 304, source data structured according to one or more other data structures (which may also be referred to herein as source data structures for convenience) are accessed for the plurality of patients in connection with the clinical trial. These data structures can be, for example, data structures like source data structures 202, 204, 206 described above in connection with FIG. 2. The source data are arranged among plural data fields of the other data structures. As mentioned previously, each data field of the other data structures may have a particular data type (e.g., integer, date, character, etc.) and may contain data of a singular, particular substantive content type (e.g., patient identification number, age, gender, height, weight, blood pressure, pre-existing medical condition, pre-existing medication, treatment medication administered, dosage, date of dosing, adverse effect, laboratory test identification, laboratory test result, date of test result, physical test identification, physical test result, date of physical test, tumor measurement, date of tumor measurement, etc., to name a few). The accessing referred to in step 304 can be carried out by a processing system by, e.g., establishing communication with one or more databases containing the other data structures, opening the other data structures, or a combination of both, for example. As shown at item 306 in FIG. 3, the other source data structures can be, e.g., a single source data structure, multiple source data structures, or a single intermediate two-dimensional array data structure generated by merging data of multiple source data structures together into one enormous two dimensional array. In this regard, such an intermediate data structure can be, e.g., intermediate data structure 226 described in connection with FIG. 2. As noted previously, the use of a single intermediate data structure 226 as described in examples herein can provide a technical advantage by reducing the communications resources and bandwidth that would otherwise be needed to communicate on an ongoing basis with a plurality of different databases and to access on an ongoing basis multiple source data structures 202, 204 and 206 potentially for which the plural databases and multiple data structures may be configured and stored according to a variety of different protocols. In other words, exemplary approaches described herein can provide a technical advantage by reducing the number of databases with which to communicate, reducing the number of source data structures to access, and reducing the number of data protocols that need to be managed, thereby increasing speed of access and processing

At step 308, desired source data for the plurality of patients are selected by the processing system from the plural data fields of the other data structures and are assigned by the processing system to the multiple data fields of the first data structure to populate the patient data of the first data structure, e.g., 230. In this example, step 308 may be carried out by the processing system according to certain rules or guidelines. First, as shown at item 310 in FIG. 3, processing can be carried out such that source data from some data fields of the other data structures are purposefully not assigned to the first data structure. As one example in this regard, laboratory test result values (e.g., for hemoglobin, white blood count, liver enzymes, etc.) that are within normal ranges are not selected and assigned to the first data structure. Rather, within the category of laboratory testing results, only those values that fall outside normal ranges are selected and assigned to the first data structure. This permits the first data structure 230 to be substantially more compact than the other source data structures containing the source data. Alternatively, instead of not assigning such data to the first data structure, certain source data could be flagged (e.g., stored with a suitable indicator or flag) so that such data could be chosen to not be displayed in a GUI window (or page), e.g., either by not populating cells of the GUI page to be visualized with such data, or by coloring the characters of such data in the GUI page using the same color as the background so that such information is not visible to a physician or other user viewing the GUI page. Or such data can be flagged so that corresponding fields of the GUI representation are presented with dot characters, hyphen characters or other unobtrusive non-numerical or non-textual characters. In either case, whether omitting certain data from the first data structure or by flagging data in the first data structure so that the associated cells of the GUI appear blank, the GUI can likewise be presented in a more compact fashion, and this can provide a technical advantage of reducing the display resources that might otherwise be required, e.g., reducing or eliminating a need for multiple display monitors, multiple video cards, or more sophisticated video cards, and the like. Moreover, this can permit displaying at the GUI the appearance of blank data cells or unobtrusive non-numerical or non-textual characters at cells of the GUI, in which case the GUI can be presented in a more compact fashion, and this can provide a technical advantage of reducing the display resources that might otherwise be required, e.g., reducing or eliminating a need for multiple display monitors, multiple video cards, or more sophisticated video cards, and the like. Other examples will be discussed in connection with FIG. 4 below.

In addition, as shown at item 312 of FIG. 3, the selecting/assigning of step 308 may comprise transforming source data of different content types for two or more fields of the other data structures into a particular, singular transformative data field of the first data structure. A transformative data field in this regard contains transformed source data of different content types from two or more source data fields, such as previously described for file 230 of FIG. 2. In other words, where the source patient data for the clinical trial contained two or more pieces of data of different content types, each in a separate data field, the first data structure, e.g., compact data structure, 230 of FIG. 2, may contain in one, single data field transformed data derived from those two or more pieces of data of different content types from the source databases. The use of transformative data fields may also provide a technical advantage of reduction in a number of fields in the first data structure and a reduction in size.

In addition, as noted at item 314 of FIG. 3, in addition to having one or more transformative data fields, the assigning of step 308 may be carried out so that first data structure contains one or more blank data fields (or coded for viewing as non-numerical or non-textual characters) that nevertheless represent substantive information, e.g., no out of range or remarkable values, such as data within a normal range or ranges, e.g., normal laboratory test values as noted above. The ability of the first data structure to represent substantive information with blank data fields can substantially enhance the compactness of the first data structure as well as ease of comprehension by a physician as will be discussed in more detail in connection with FIG. 4. In this way, the GUI can be presented in a more compact fashion, and this can provide a technical advantage of reducing the display resources that might otherwise be required, e.g., reducing or eliminating a need for multiple display monitors, multiple video cards, or more sophisticated video cards, and the like.

Step 308 can be carried out in various ways. For example, as shown at item 316 of FIG. 3, the selecting and assigning of step 308 can be carried out content type by content type for all patients, one after the other. In other words, a given content type of data, such as patient ID (i.e., an assigned identification number that does not reveal a patients' true identity), can be selected for all patients and then assigned to the first data structure. Then a next content type, such as date of birth, can be selected for all patients and then assigned to the first data structure. This process can be repeated until all desired data has been selected from the source data structures and assigned to the first data structure. In an alternative example, as shown at item 318, the selecting and assigning can be carried out patient by patient for all content types one after another. In other words, all desired data of all content types can be selected for a first patient, and all of that desired data for the first patient can be assigned to the first data structure. This process can then be repeated for the next patient until all desired patient data has been selected and assigned to the first data structure 230.

As noted above, the first data structure 230 can be, for example, a two-dimensional array data structure suitable for displaying data fields for visualization via a two-dimensional GUI page 400, such as in an example of a two-dimensional spreadsheet of rows and columns. The first data structure 230 can be structured for example as a hash map, or alternatively as a linked list (e.g., a linked list of linked lists) or in any other suitable manner. In addition, the size of the first data structure 230 may be reduced in size (e.g., in terms of megabytes, gigabytes, etc.) by 50%, 60%, 70%, 80%, or more compared to the size of the source data for the plurality of patients in the data source structures 202, 204, 206. For example, a typical size of patient data for a clinical trial involving 1000 patients stored in multiple source data structures 202, 204, 206 of several different databases may occupy about 1.1 gigabytes of memory storage, for instance, whereas the amount of patient data stored in the first, compact data structure 230 can be of reduced size to occupy, for instance, about 25 megabytes, of memory storage. Where an intermediate data structure 226 is additionally utilized, its size may occupy, for instance, about 0.2 gigabytes of memory storage. It should be noted that these exemplary reductions in size do not take into account any additional size reductions that may be obtained through computerized data compression algorithms, such as compression algorithms conventionally known to those of ordinary skill in the art.

A listing of exemplary guidelines that may apply to the selection, transformation and assignment of patient data to the first data structure may include, for example, any, all, or any combinations of portions of the following:
laboratory result data, e.g., white blood cell count, that fall within normal ranges are not to be stored in the first data structure,
laboratory result data, e.g., white blood cell count, that fall outside normal ranges are to be stored in the first data structure,
laboratory result data that fall outside the normal range may be reported along with the upper or lower or end of the normal range as applicable,
physical test data e.g., tumor size measurements, are to be stored in the first data structure,
dosages and identifications and dates of administration of test drugs are to be stored in the first data structure,
concomitant medication information for administered drugs are to be stored in transformative data fields that include in a singular data field multiple content types of data including drug name, date of administration, and whether or not the administration of the drug is to be ongoing,
adverse event information is to be stored in the first data structure,
adverse event information is to be stored in transformative data fields that include in a singular data field multiple content types of data including the adverse event number, adverse event name, date of onset of the adverse event, one or more additional dates of assessment of the adverse event, date of resolution of the adverse event, outcome of the adverse event, whether the adverse event resulted in a change in medication assignment or dose, whether the adverse event required hospitalization and whether the adverse event was suspected to be related to a particular medication.

As shown at step 320 of FIG. 3, patient data of the first data structure is processed by the processing system for visualization at a graphical user interface such that representations of the multiple data fields of the first data structure for an individual patient are arranged for display via a single window (which may also be called a page) of the graphical user interface (GUI). An example will be described below with reference to FIGS. 4A-4Q.

FIG. 4A illustrates an exemplary system for processing and displaying patient medical data according to the approaches described herein. The system shown in FIG. 4A includes a computer system 410 that includes a processing system (e.g., one or more computer processors) that stores and processes a first data structure such as previously described, one or more source data structures DS1-DSN 420 which may be processed to generate the first data structure, and a display system 430 for viewing a GUI page for visualizing data of the first data structure. In FIG. 4A, perimeter dashed line denotes schematically an exemplary GUI page 400 (or window), which, merely for ease of description, is shown in FIG. 4A as a collection of tiled rectangles shown by dashed lines in vertical columns and horizontal rows. One vertical column of such tiles is labeled 400-B, 400-C, 400-D, 400-E, etc., and one horizontal row of such tiles is labeled 400-E, 400-N, 400-O, 400-P, etc. Exemplary details regarding characteristics of the GUI page 400 will be described in greater detail below with reference to FIGS. 4B-4Q, each of which corresponds to a particular labeled tile shown in FIG. 4A, for convenience.

FIGS. 4B-4Q illustrate individual portions of the exemplary page 400 (outlined in FIG. 4A) of a graphical user interface (GUI) where patient data for a single patient receiving treatment for a disease in a clinical trial (e.g., drug treatment for lymphoma in this example) is displayed in a two-dimensional array format according to the first data structure. In this example, a given field of the GUI page 400, as well as a given field of the first data structure, can be represented by a first set of indices (e.g., column identifications, such as letters) and a second set of indices (e.g., row identifications, such as numbers). An example is the heading “SCREENING” shown in FIG. 4B at field (or cell) A2 (i.e., column A, row 2) of portion 400-B of GUI page 400. The patient data illustrated in the example of FIGS. 4B-4Q (as well as that in FIG. 5) is fictitious—it does not represent clinical data for a real patient. While the example of FIGS. 4B-4Q is presented in the context of treatment for lymphoma, the systems and approaches described herein are in no way limited to the example of lymphoma and are generally applicable to the processing and visualization of patient data in connection with medical treatments generally as well as clinical trials for other diseases, treatments, products, devices etc. Data for multiple other patients may be navigated and presented for viewing via other similar pages 400 of the GUI, e.g., such as via a separate tab for each individual patient which when clicked via a computer mouse brings a single GUI page 400 for that patient to the forefront of the GUI for viewing.

The exemplary GUI page 400 illustrated in FIGS. 4B-4Q, while divided among multiple drawing sheets (tiled as illustrated in FIG. 4A) for convenience and ease of illustration for purposes of this disclosure, is in fact a single page of the GUI. It will be understood that the single page of GUI page 400 is viewable by adjusting magnification of the information displayed using a suitable selection via the GUI by a user and by scrolling to the left or right and up and down using a computer workstation, e.g., user PC with a touch screen or computer mouse, as would be understood by one of ordinary skill in the art. As noted previously, the transformed patient data stored in the first (e.g., compact) data structure may include multiple sets of data fields, which are likewise reflected in the GUI page 400, which will now be described for the example of FIGS. 4B-4Q.

The GUI page 400, as well as the first data structure, includes a first set of data fields for “screening” information for the given patient. These data fields are illustrated generally in rows 2-67 of the GUI page 400 in FIGS. 4B-4E, starting with the heading “SCREENING” in field A2. As shown in FIGS. 4A-4C, the screening information includes information such as ID (identification) at field (or cell) B3, randomization number (RAND), which represents the randomized number of the patient in the study, at field B4, the patient's initials (INI) at field B 5, age at field B6, gender at field B 7, date of birth (DOB) at field B8, consent date (CONSENTDT) at field B9, randomization date (RANDDT) at field B10, patient weight at field B11, patient height at field B12, BSA at field B13, patient blood pressure at field B14, patient pulse at field B15, patient temperature at field B16, etc. The screening information may also include medical history information, which is shown below the heading “MEDX” at field B25. This information may include, for example, data for whether there has been a prior venous thromboembolism (VTE) deep vein thrombosis (DVT) at field B26, a VTE pulmonary embolism (PE) at field B27, or other prior VTE at filed B28.

Other medical history information is shown at fields B30-B36, any of which may represent a transformed data field such as described above. For instance, in the example of FIG. 4C, field B30 reflects that the patient has a medical history of GERD (gastroesophageal reflux disease), as of November 2011 (112011), and that the condition is ongoing. This field represents an example of a transformative data field in so far as field B30 contains both a reference to GERD, the date 112011, and the fact that the condition is ongoing, and therefore contains data of several different substantive content types, namely, an identification of the condition, date information as well as status information for the pre-existing condition (ongoing). This data field is transformative since, for example, each of those three pieces of data existed in separate data fields of singular content type and/or data type in the source databases from which this information was derived. Moreover the fact that this data field of the first data structure 230 is transformative is further highlighted, for example, by the fact that the transformation is not reversible in the sense that it cannot be undone by a one-to-one mapping of one set of data fields of particular content types of one data structure to another set of data fields of particular content types of another data structure.

The compactness of the first data structure 230 can be enhanced, for instance, as shown by the example of FIGS. 4B-4Q, by utilizing extensive use of abbreviations. In the particular example for lymphoma treatment reflected in FIGS. 4B-4Q, any abbreviations not specifically described herein would be readily recognized by one of ordinary skill in the art. For purposes of illustration of the descriptions of various data, the GUI page 400 illustrated in the excerpts 400-B through 400-Q in FIGS. 4B-4Q displays the full contents of each cell. However, the compactness of the first data structure may also be enhanced by presenting data in some or all of the data fields in a single line as shown in the example of FIG. 5, as opposed to displaying the full contents in each field such in multiple lines. FIG. 5 shows an exemplary excerpt of the GUI page 400 labeled 400-EF, since the data in FIG. 5 corresponds to that shown in FIG. 4E (excerpt 400-E) and FIG. 4F (excerpt 400-F). As shown in the example of FIG. 5, in such an instance, the full contents of any given data field may be expanded for observation by simply moving a mouse guided cursor above any given data field and hovering at that point so as to generate a pop-up window showing the full contents of the data field such as shown at numeral 450, or by clicking on a data field such that the full contents are observable in another display portion of the GUI page 400.

The screening information may also include information regarding concomitant medications, such as illustrated in rows 38-40 of FIG. 4C, as well as information regarding disease history, such as shown below the heading “LYMPHOMA HISTORY” at rows 42-67 of FIGS. 4D and 4E. In this example, the data fields B38-B40 of FIG. 4C are transformative data fields containing the names of the medications, beginning date of use of the medication, and indications that the use is ongoing. Those data fields could include, depending upon circumstances, an indication that the medication use has terminated and a date of termination of use. Disease history information, such as shown at B42-B67 of FIGS. 4D and 4E, for example, may include any suitable information, including blanks for certain data fields, such as B54 (GELF+LDH Beta 2) and B64 (LDH), for which there is no applicable data, or for which the associated data is within a normal range. In some examples, blank fields may be used where data is absent for such fields, and fields presented with dot characters, hyphen characters or other unobtrusive non-numerical and non-textual characters may be used to represent data that are unremarkable or within normal ranges. Alternatively, in some examples, fields presented with dot characters, hyphen characters or other unobtrusive non-numerical and non-textual characters may be used where data is absent for such fields, and blank fields may be used to represent data that are unremarkable or within normal ranges. In, either of these examples, at “Toggle” function may be used, shown by a “Toggle” button near the tops of FIGS. 4B-4Q and 5, whereby data in normal ranges or which is otherwise unremarkable may be “hidden” from view at the GUI in one state but which may be shown for viewing at the GUI by clicking or selecting the “Toggle” button. In this way, a user may switch between viewing a minimal view, where normal-range data and otherwise unremarkable data are hidden and not shown on the GUI, to a maximal view, where such “hidden” data is presented for viewing at the GUI. Also, a variety of other views may be selected via buttons labeled View 1, View 2, View 3, View 4, etc., FIGS. 4B-4Q and 5. These views are user customizable views that permit a user to set up and select between views that a user may find particularly desirable, e.g., such as displaying all laboratory values including normal values but not displaying other unremarkable data, displaying data only selected types of data, displaying only data from a certain time period(s) or cycle(s), etc., or any desired combinations. These views may be set up, e.g., via a configuration menu under the “Tools” menu, whereby certain views can be defined by selecting desired fields with check boxes or any other suitable means. The first data structure can be coded in such a way that certain data, e.g., normal-range data, unremarkable data, etc., is distinguishable from other data of significant interest. Thus, the first data structure may comprise data coded or flagged for different viewing options based on the substantive value(s) of the actual data, and not simply based on the field name or other attribute of the data. In this way, the first data structure may contain as much patient data is desired, including substantially complete patient data, but the display of the data can be tailored so that the physician need not view a screen laden with a vast amount of distracting, unremarkable data. The screening fields may also include transformative data fields, such as described above, if applicable.

As shown in FIG. 4E, the GUI page 400, as well as the first data structure, also includes a second set of data fields for “treatment” information for the given patient. These are illustrated generally in rows 69-79 of the GUI page 400 in FIG. 4E, starting with the heading “TREATMENT” in field A69. As shown in FIG. 4E, the treatment information includes information such as cycle date (row 70), whether or not the treatment was interrupted for some reason, e.g., with reference to an adverse event number (row 71, e.g., data field V71 in FIG. 40), particular treatment drug and dosage, e.g. Rituxan (row 72), etc.

As shown in FIG. 4F, the GUI page 400, as well as the first data structure, also includes a third set of data fields for information regarding patient response to treatment for the given patient. These are illustrated generally in rows 81-99 of the GUI page 400 in FIG. 4F, starting with the heading “RESPONSE FOLLOWING TREATMENT” in field A81. As shown in FIG. 4F, the information regarding response to treatment includes information such as clinical exam (row 82), ECOG (row 83), LDH (row 84), B symptoms (row 85), bone marrow biopsy (row 86), PET (row 87), extra nodal evaluable but unmeasurable (row 88). The response to treatment information may also include physical measurements e.g. such as tumor size measurements. For example, rows 90-95 of FIG. 4F reflect tumor size measurements for target lesions on a particular date Jun. 29, 2012 (29062012). As noted previously, a general guideline for recording such data is to include physical measurements in the first data structure 230 and to display such values in the GUI page 400. Various other information relating to response to treatment is also shown in this section.

As shown in FIGS. 4G-4H, the GUI page 400, as well as the first data structure, includes a fourth set of data fields for information regarding laboratory information to treatment for the given patient. These are illustrated generally in rows 101-127 of the GUI page 400 in FIGS. 4G-4H, starting with the heading “LABORATORY VALUES” in field A101. As shown in FIGS. 4G-4H, the laboratory results information includes information such as hemoglobin (row 102), white blood cell count (WBC) (row 103), ANC (row 104), ALC (row 105), monocytes (row 106), platelets (row 107), RBC (row 108), hematocrit (row 109), AST (110), ALT (row 111), alkaline phosphatase (row 112), total bilirubin (row 113), LDH (row 114), etc. As described previously, in some examples, laboratory result values that are within normal ranges need not be stored in the first data structure or displayed in the GUI page 400, and the corresponding blank spaces nevertheless represent substantive information, e.g., that such data are within normal ranges or are unremarkable. In this regard, blank fields may be used to represent data that are unremarkable or within normal ranges, and fields presented with dot characters, hyphen characters or other unobtrusive non-numerical and non-textual characters may be used to represent data that are missing or absent. Alternatively, in some examples, fields presented with dot characters, hyphen characters or other unobtrusive non-numerical and non-textual characters may be used to represent data that are unremarkable or within normal ranges, and blank fields may be used to represent that data are missing or absent. Laboratory result values that are not within normal ranges, however, are stored in the first data structure and displayed in the GUI page 400. According to one aspect, such data outside normal range may be displayed along with the normal-range value that is exceeded. Such an example is shown, for instance, at field B103 for WBC with entry “3.90 v 4.00” which conveys that the laboratory result value is 3.90, and it is outside the normal range on the low side, the low-side normal value being 4.00. It will be appreciated that this entry reflects another example of a transformative data field, insofar as it contains the information regarding the substantive laboratory value and information regarding the normal range for the particular property being tested.

In a variation on this example, the appearance and benefit of blank space as discussed above can also be obtained in the following manner or variations thereof. In particular, as mentioned previously, instead of not assigning certain data to the first data structure from the source data structures, certain source data could be flagged (e.g., stored with a suitable indicator or flag) so that such data could be chosen to not be displayed in the GUI page 400, e.g., either by not populating cells of the GUI page to be visualized with such data, or by coloring the characters of such data in the GUI page using the same color as the background so that such information is not visible to a physician or other user viewing the GUI page. For instance, more source data or possibly all source data from the source data structures, including laboratory result data within normal ranges, could be stored in the first data structure. But certain flagged data, such as laboratory results within normal ranges, could be processed by the processing system so as to not be displayed in the GUI 400 page. Alternatively, such flagged data could be populated into the GUI page 400 as well, but coded in characters of the same color as the background, e.g., white, so that they would not be visible to the viewing physician. That flagged information, however, could still be accessible for viewing by the physician via the GUI, e.g., by hovering the cursor over a cell normally displayed in the GUI as “white” or “blank” such that the data associated with that cell then becomes visible at the GUI page 400 via a popup window, such as shown at numeral 450 in FIG. 5. In this way, the processing system may process data of the first data structure such that certain data fields of the multiple fields of the first data structure are structured so as to not visibly convey numerical or textual data via the GUI 400, wherein at least some of the certain data fields convey substantive information by virtue not displaying visible information. And as noted above, in some examples, blank fields may be used to represent data that are unremarkable or within normal ranges, and fields presented with dot characters, hyphen characters or other unobtrusive non-numerical and non-textual characters may be used to represent data that are missing or absent. Alternatively, in some examples, fields presented with dot characters, hyphen characters or other unobtrusive non-numerical and non-textual characters may be used to represent data that are unremarkable or within normal ranges, and blank fields may be used to represent that data are missing or absent.

As shown in FIGS. 4I-4J, the GUI page 400, as well as the first data structure, also includes a fifth set of data fields for “adverse event” information for the given patient. These are illustrated generally in rows 129-139 of the GUI page 400 in FIGS. 4I-4J starting with the heading “SAEs” in field A129. As shown in FIGS. 4I-4J, the adverse events information includes information for both serious adverse events (SAEs) (row 129) as well as other adverse events (AEs) (rows 131-139) separated by the adverse event's grade, AE 4& 5 (row 131), AE 3 (row 133), AE 1 & 2 (row 135-139). A particular example of an adverse event as reflected in the example of FIG. 4I is shown at field D136, which shows that adverse event number 4, i.e., nausea, for which the first date of Aug. 24, 2012 (24082012) is the date of worst grade, the second date of Aug. 24, 2012 is the start date of the adverse event, and the third date of Apr. 19, 2013 (19042013) is the date of last follow-up for which the adverse event was resolved without sequelae. This data field also presents a further example of a transformative data field in so far as it presents not only information about the type of adverse event, but the date on which the adverse event occurred, the date on which the adverse event reached it's worse grade, the date on which the AE was resolved, and the outcome of the adverse event, which is a recovery in this example. Such as described previously, all of these types of information would have existed in separate singular data fields of different content type and data type in the source databases and data structures.

As shown in FIGS. 4K-4L, the GUI page 400, as well as the first data structure, also includes a sixth set of data fields for “medication” information for the given patient. These are illustrated generally in rows 140-152 of the GUI page 400 in FIGS. 4K-4L, starting with the heading “CONCOMITANT MEDICATIONS” in field A140. As shown in FIGS. 4K-4L, the medication information may generally be represented using transformative data fields, in particular, data fields that include the name of the medication, the date of administration, and whether or not the medication was administered on an ongoing basis. As an example, field C144 reflects that Aspirin was administered in this example on Jul. 6, 2012 (06072012) and that administration of the drug is ongoing at the end of the cycle. This transformative data field contains several different content types of information, namely, the name of the medication, the date it was administered, and whether it's administration is ongoing. Such as described previously, these three different pieces of information would have been stored in singular separate data fields in the source databases having singular content types and data types.

Additional array field headings and associated data for the GUI page 400, as well as the first data structure, are shown in FIGS. 4M-4Q, e.g., at column headings at fields I1-AL1. The field headings shown in FIGS. 4M-4Q designate particular cycles and other time-based events for representing the information shown in rows 2-152 for the various cycles and events of the clinical trial example. FIGS. 4M-4Q shows data of rows 58-79 from Cycle 6 through Cycle 24 and through further Evaluation and Follow up events. Data such as that previously described with regard to the FIGS. 4A-4L may also exist throughout this single GUI page 400 for these additional array headings shown in FIGS. 4M-4Q.

The first data structure 230 as described herein may provide a number of advantages for efficiently processing and visualizing patient data in clinical trials. For example, the first data structure 230 as described herein may store clinical trial data for a plurality of patients structured as two-dimensional array, with at least some fields of the first data structure containing transformed data in transformative data fields that combines two or more content types into a single data field, and wherein some data fields are blank but nonetheless convey substantive information, such as patient data within a normal range. Also, as noted above, in some examples, blank fields may be used to represent data that are unremarkable or within normal ranges, and fields presented with dot characters, hyphen characters or other unobtrusive non-numerical and non-textual characters may be used to represent data that are missing or absent. Alternatively, in some examples, fields presented with dot characters, hyphen characters or other unobtrusive non-numerical and non-textual characters may be used to represent data that are unremarkable or within normal ranges, and blank fields may be used to represent that data are missing or absent. Moreover, this patient data can be formatted for presentation and visualization such that the patient data of the first data structure 230 is displayed on a single GUI page, such as GUI page 400 of FIGS. 4B-4Q, without inundating the user, e.g., physician, with enormous amounts of unneeded text, descriptors, normal-range data, etc., that could otherwise cloud or obscure more important data that should be presented to the physician. As can be readily appreciated from inspection of FIGS. 4B-4Q, the single GUI page 400 comprises substantial “white space” so that the physician or other decision-maker is presented with the most desired data in numerical, character, date, or other form, without being inundated with a densely populated report comprising vast amounts of unremarkable information including ordinary descriptions and normal-range data. Thus the single GUI 400 is easy to view and comprehend and presents a substantial time savings and efficiency enhancement for physicians and other decision-makers who must review and comprehend clinical trial data for a given patient. Indeed, experimental analyses based on visualization of patient data structured according to a two-dimensional array as described herein, have indicated efficiency enhancements of 70% or more in terms of time reductions required by physicians or other decision makers to clean, analyze and comprehend patient data, when structured according to the approaches described herein.

Moreover, the use of an intermediate data structure, such as intermediate data structure 226 of FIG. 2, in which all of the source data of the exemplary source data structures 202, 204, 206 is merged into one relatively large, intermediate two-dimensional array data structure 226, can provide a technical advantage of processing simplicity and speed. In particular, the use of one large, intermediate two-dimensional array data structure 226 can provide an efficient means for writing desired source data of various data fields to the first data structure 230 of FIG. 2, through use of computer code that need only access the single, intermediate data structure 226, as opposed to multiple different databases and data structures, 202, 204, 206. This can provide a technical advantage by reducing the communications resources and bandwidth that would otherwise be needed to communicate on an ongoing basis with a plurality of different databases comprising multiple source data structures 202, 204 and 206 potentially stored according to different protocols. In addition the use of an intermediate data structure 226 provides additional ease of preparing graphs and statistics based on the clinical trial data (patient data), since all of the data that underlies the clinical trial data for the plurality of patients exists in the single intermediate data structure, and need not be accessed from multiple different data structures in different databases. Thus, while the use of an intermediate data structure 226 in the form of a large two-dimensional array of clinical trial data as described herein may be of little use itself for directly visualizing and comprehending patient data directly by a physician, and is considered contrary to the conventional wisdom, the present inventors have observed and appreciated that the use of such an intermediate data structure 226 containing substantially complete patient data for the entire clinical trial in a single two-dimensional array may provide for efficiencies such as described above by reducing the number of databases with which to communicate, reducing the number of source data structures to access, reducing the number of data protocols that need to be managed for such, and thereby increasing speed of access and processing.

In another example representing a variation on the first data structure and GUI page 400 described above, the first data structure and the GUI page 400 can be structured so that multiple first group of multiple columns of patient data (e.g., for certain cycles, assessments or other time-based events) can be viewed on a first GUI page, and a second group of multiple columns of patient data (e.g., for certain cycles, assessments or other time-based events) can be viewed on a second GUI page. For example the first GUI page could be selected via a first tab at the bottom of the GUI display, and the second GUI page could be selected via a second tab at the bottom of the GUI display. Moreover, the GUI may include functionality that permits both the first GUI page and the second GUI page to be resized such that they can be viewed simultaneously side-by-side on a display system. In other examples, different cycles could be individually selectable for viewing via the GUI. In a further example representing a variation on the first data structure and GUI page 400 described above, additional data such as some or all normal-range laboratory-result data could be included in the first data structure and viewable via GUI page 400, but certain such data, such as laboratory result data that is out of normal range, could be flagged, e.g., so as to be highlighted in color (e.g., yellow) on the GUI page 400 to focus attention on the flagged, e.g., out-of-normal data.

In other aspects, the patient information as described in the examples herein has been preprocessed and de-identified to remove patient identifying information, such as patient name, social security number, address, or other identifying information, at an early stage of data processing in the clinical trial. In particular, the patient data stored in either or both of the source data structures 202, 204, 206, or the intermediate data structure 226, is already de-identified as stored in those data structures. This de-identification removes the patient identifying information except for a numerical patient ID associated with each patient, which the physicians and other medical professionals associated with the clinical trial cannot correlate to the patients' names, social security numbers, addresses, or other actual identifying information. This de-identification protects both the integrity of the clinical trial and the privacy of the patients (subjects) participating in the clinical trial. Such de-identification may be carried out to be commensurate with the Health Insurance Portability and Accountability Act (HIPPA) so as to satisfy federal regulations relating to the privacy and confidentiality and consents, authorizations, and notices relating to patient information.

The data structures as described herein may utilize naming conventions such as those associated with the Clinical Data interchange Standards Consortium (CDISC) Operational Data Model (ODM), which is a data model designed to facilitate the regulatory-compliant acquisition, archive and interchange of metadata and data for clinical research studies, as is known to those of ordinary skill in the art, the entire contents of which are incorporated herein by reference. The ODM is a platform-independent format for exchanging and archiving clinical study data that is non-specific to any given database vendor. Where medical devices are implicated, the data structures as described here may also utilize naming conventions such as described in the (CDISC) Study Data Tabulation Model (SD™) Implementation Guide for Human Clinical Trials, e.g., Version 3.2, the entire contents of which are incorporated herein by reference. However, the first data structure as described herein is very different from any data models associated with the SDTM or ODM. As described above, for example, the first data structure described herein, stores clinical trial data for a plurality of patients whereby the clinical data for a given patient is structured as two-dimensional array, with at least some fields of the first data structure containing transformed data that combines two or more content types into a single data field and wherein some data fields are blank but nonetheless convey substantive information, such as patient data within a normal range. Also, as noted above, in some examples, blank fields may be used to represent data that are unremarkable or within normal ranges, and fields presented with dot characters, hyphen characters or other unobtrusive non-numerical and non-textual characters may be used to represent data that are missing or absent. Alternatively, in some examples, fields presented with dot characters, hyphen characters or other unobtrusive non-numerical and non-textual characters may be used to represent data that are unremarkable or within normal ranges, and blank fields may be used to represent that data are missing or absent. Moreover, this patient data can be formatted for presentation and visualization such that the patient data of the first data structure is displayed on a single GUI page, such as GUI page 400 of FIGS. 4B-4Q, without inundating the user, e.g., physician, with enormous amounts of ordinary, uncontroversial information and normal-range data. Conventional approaches do not structure or present patient data in this novel manner.

FIG. 6 illustrates a block diagram of an exemplary computer environment 600, a networked client-server environment, for use in transforming source patient data formatted according to one or more computer data structures into a different format of a different, first computer data structure according to an to an aspect of the present disclosure. In the exemplary computer environment 600 illustrated in FIG. 6, users, via user computers or workstations (e.g., PCs) 602, can interact with a computer processing system 604 running a transformation engine 606, which may be hosted on one or more computer servers 610 through one or more networks 608. The transformation engine 606 contains software operations or routines to implement the functionality described herein. The user computers 602 can interact with the transformation engine 606 through a number of ways, such as over one or more networks 608. One or more servers 610 accessible through the network(s) 608 can host transformation engine 606, which may access non-transitory computer readable memory 612. It should be understood that the transformation engine 606 could also be provided on a stand-alone computer for access by a user. The transformation engine 606 may access source data 616 for a plurality of patients contained in one or more data structures, e.g., in data store(s) 614, transform the source data to provide another, e.g., compact representation, and store transformed data 618 for the plurality of patients in a first data structure, e.g., in data store(s) 614 as previously explained herein. For example, a single data field of the first data structure 230 may contain consolidated data of more than content type. Thus, the transformation engine 606 can provide a technical advantage of generating data fields that permit the first data structure 230 to be reduced in size, thereby lessening storage resources required for the first data structure 230 as well as a technical advantage of permitting the GUI display to be more compact, thereby reducing the display resources that might otherwise be required, e.g., reducing or eliminating a need for multiple display monitors, multiple video cards, or more sophisticated video cards, and the like.

The processing system 604, e.g., one more computer processing units (CPUs) (central processing unit), such as that described in connection with FIG. 2, may perform calculations and logic operations required to execute a program. A non-transitory computer-readable storage medium 612, such as read only memory (ROM), random access memory (RAM), flash memory, optical or magnetic disk, or other nonvolatile memory or non-transitory physical storage medium, may be in communication with the processing system 604 and may contain one or more programming instructions. Computer instructions may also be communicated via a communications signal, or a modulated carrier wave, e.g., such that the instructions may then be stored on a non-transitory computer-readable storage medium. Element managers, real-time data buffers, conveyors, file input processors, database index shared access memory loaders, reference data buffers and data managers may each include one or more software applications stored in one or more computer memories, and the processing system 604 may access each component as required.

The methods and systems described herein may be implemented on many different types of processing devices by program code comprising program instructions that are executable by the device processing system. The software program instructions may include source code, object code, machine code, or any other stored data that is operable to cause a processing system to perform the methods and operations described herein. Any suitable computer languages may be used such as C, C++, Java, etc., along with any native commands associated with database systems being utilized, e.g., SAS databases, as will be appreciated by those skilled in the art. Other implementations may also be used, however, such as firmware or even appropriately designed hardware configured to carry out the methods and systems described herein.

Data (e.g., associations, mappings, data input, data output, intermediate data results, final data results, etc.) associated with the systems and methods described herein may be stored and implemented in one or more different types of computer-implemented data stores, such as different types of storage devices and programming constructs (e.g., RAM, ROM, Flash memory, flat files, databases, programming data structures, programming variables, IF-THEN (or similar type) statement constructs, etc.). It is noted that data structures describe formats for use in organizing and storing data in databases, programs, memory, or other non-transitory computer-readable media for use by a computer program.

The computer components, software modules, functions, data stores and data structures described herein may be connected directly or indirectly to each other in order to allow the flow of data needed for their operations. It is also noted that a module or processor includes but is not limited to a unit of code that performs a software operation, and can be implemented for example as a subroutine unit of code, or as a software function unit of code, or as an object (as in an object-oriented paradigm), or as an applet, or in a computer script language, or as another type of computer code. The software components and/or functionality may be located on a single computer or distributed across multiple computers depending upon the situation at hand.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps. It should also be understood that as used in the description herein and throughout the claims that follow, the meaning of “a,” “an,” and “the” includes plural reference unless the context clearly dictates otherwise. Also, as used in the description herein and throughout the claims that follow, the meaning of “in” includes “in” and “on” unless the context clearly dictates otherwise. Finally, as used in the description herein and throughout the claims that follow, the meanings of “and” and “or” include both the conjunctive and disjunctive and may be used interchangeably unless the context expressly dictates otherwise.

While exemplary embodiments have been shown and described herein, it will be appreciated by those skilled in the art that such embodiments are provided by way of example only. It is intended that the following claims define the scope of the invention and that methods and structures within the scope of these claims and their equivalents be covered thereby.

Any discussion of documents, acts, materials, devices, articles or the like which has been included in the present specification is not to be taken as an admission that any or all of these matters form part of the prior art base or were common general knowledge in the field relevant to the present invention as it existed before the priority date of each claim of this application.

1. A computerized method of transforming patient data formatted according to one or more computer data structures into another format of another computer data structure for efficient processing and visualization of patient data, the method comprising:
defining a first data structure comprising multiple data fields in which to store patient data for a plurality of patients;
accessing source data structured according to one more other data structures for the plurality of patients, the source data being arranged among plural data fields of the one or more data structures, each of the plural data fields of the one or more other data structures having a data type, each of the plural data fields of the one or more other data structures having a content type, the one or more other data structures being different in structure from the first data structure;
selecting desired source data for the plurality of patients from the plural data fields of the one or more other data structures;
assigning selected source data from the plural data fields of the one or more other data structures to the multiple data fields of the first data structure to populate the patient data of the first data structure,
wherein the assigning includes transforming source data of different content types for two or more fields of the one or more other data structures into a particular transformative data field of the first data structure,
wherein the multiple data fields of the first data structure include data fields for multiple treatment cycles at different times; and
processing the patient data of the first data structure for visualization at a graphical user interface such that representations of fields of the multiple data fields of the first data structure for the given patient are arranged for display via a single window of the graphical user interface, and such that certain fields of the multiple fields of the first data structure are structured so as to not visibly convey numerical or textual data via the graphical user interface, wherein at least some of the certain fields convey substantive information by virtue of not displaying visible numerical or textual data.

2. The method of claim 1, wherein assigning selected source data from the plural data fields comprises not assigning source data from some of the plural data fields of the one or more data structures.

3. The method of claim 1, wherein the multiple data fields of the first data structure for a given patient include a first set of data fields for storing screening information for the given patient, a second set of data fields for storing treatment information for the given patient, a third set of data fields for storing information regarding patient response to treatment for the given patient, a fourth set of data fields for storing laboratory results information for the given patient, a fifth set of data fields for storing medication information for the given patient, and a sixth set of data fields for storing adverse event information for the given patient; and
wherein the second, third, fourth, fifth, and sixth sets of data fields include data fields for multiple treatment cycles at different times.

4. The method of claim 1, wherein after said assigning, some of the multiple fields of the first data structure are blank data fields containing no numerical or textual data, wherein at least some of the blank data fields convey substantive information such that patient data corresponding to such fields are within a normal range or unremarkable by virtue of being represented a blank data fields, and wherein some other fields of the first data structure are processed to be visualized at the graphical user interface with a non-numeric, non-textual character(s) to convey that source data are missing or absent for such fields.

5. The method of claim 1, wherein after said assigning, some of the multiple fields of the first data structure are blank data fields for which source data are absent or missing, and wherein at least some other fields of the first data structure are processed to be visualized at the graphical user interface with non-numeric, non-textual characters which convey substantive information such that patient data corresponding to such fields are within a normal range or unremarkable by virtue of being represented by non-numeric, non-textual characters.

6. The method of claim 1, the first data structure comprising the patient data for the plurality of patients is reduced in size by at least 50% compared to the one or more data structures comprising the source data for the plurality of patients, said reduction in size being achieved without additional computerized data compression algorithms.

7. The method of claim 1, wherein the one or more data structures comprises multiple data structures, and wherein said assigning comprises merging the source data of the multiple data structures into a second data structure configured as a two dimensional array.

8. The method of claim 1, comprising not selecting for assignment source data from the one or more other data structures that comprises laboratory test data with a normal range.

9. A computer system for transforming patient data formatted according to one or more computer data structures into another format of another computer data structure for efficient processing and visualization of patient data, the method comprising:
a processing system; and
a memory coupled to the processing system,
wherein the processing system is configured to execute steps comprising:
defining a first data structure comprising multiple data fields in which to store patient data for a plurality of patients;
accessing source data structured according to one more other data structures for the plurality of patients, the source data being arranged among plural data fields of the one or more data structures, each of the plural data fields of the one or more other data structures having a data type, each of the plural data fields of the one or more other data structures having a content type, the one or more other data structures being different in structure from the first data structure;
selecting desired source data for the plurality of patients from the plural data fields of the one or more other data structures;
assigning selected source data from the plural data fields of the one or more other data structures to the multiple data fields of the first data structure to populate the patient data of the first data structure,
wherein the assigning includes transforming source data of different content types for two or more fields of the one or more other data structures into a particular transformative data field of the first data structure,
wherein the multiple data fields of the first data structure include data fields for multiple treatment cycles at different times; and
processing the patient data of the first data structure for visualization at a graphical user interface such that representations of fields of the multiple data fields of the first data structure for the given patient are arranged for display via a single window of the graphical user interface, and such that certain fields of the multiple fields of the first data structure are structured so as to not visibly convey numerical or textual data via the graphical user interface, wherein at least some of the certain fields convey substantive information by virtue of not displaying visible numerical or textual data.

10. The system of claim 9, wherein assigning selected source data from the plural data fields comprises not assigning source data from some of the plural data fields of the one or more data structures.

11. The system of claim 9 or 10, wherein the multiple data fields of the first data structure for a given patient include a first set of data fields for storing screening information for the given patient, a second set of data fields for storing treatment information for the given patient, a third set of data fields for storing information regarding patient response to treatment for the given patient, a fourth set of data fields for storing laboratory results information for the given patient, a fifth set of data fields for storing medication information for the given patient, and a sixth set of data fields for storing adverse event information for the given patient; and
wherein the second, third, fourth, fifth, and sixth sets of data fields include data fields for multiple treatment cycles at different times.

12. The system of claim 9, wherein after said assigning, some of the multiple fields of the first data structure are blank data fields containing no numerical or textual data, wherein at least some of the blank data fields convey substantive information such that patient data corresponding to such fields are within a normal range or unremarkable by virtue of being represented a blank data fields, and wherein some other fields of the first data structure are processed to be visualized at the graphical user interface with a non-numeric, non-textual character(s) to convey that source data are missing or absent for such fields.

13. The system of claim 9, wherein after said assigning, some of the multiple fields of the first data structure are blank data fields for which source data are absent or missing, and wherein at least some other fields of the first data structure are processed to be visualized at the graphical user interface with non-numeric, non-textual characters which convey substantive information such that patient data corresponding to such fields are within a normal range or unremarkable by virtue of being represented by non-numeric, non-textual characters.

14. The system of claim 9, the first data structure comprising the patient data for the plurality of patients is reduced in size by at least 50% compared to the one or more data structures comprising the source data for the plurality of patients, said reduction in size being achieved without additional computerized data compression algorithms.

15. The system of claim 9, wherein the one or more data structures comprises multiple data structures, and wherein said assigning comprises merging the source data of the multiple data structures into a second data structure configured as a two dimensional array.

16. The system of claim 9, wherein the processing system is configured to not select for assignment source data from the one or more other data structures that comprises laboratory test data with a normal range.

17. A non-transitory computer readable medium for transforming patient data formatted according to one or more computer data structures into another format of another computer data structure for efficient processing and visualization of patient data, non-transitory computer readable medium comprising computer instructions which when executed cause a processing system to execute steps comprising:
defining a first data structure comprising multiple data fields in which to store patient data for a plurality of patients;
accessing source data structured according to one more other data structures for the plurality of patients, the source data being arranged among plural data fields of the one or more data structures, each of the plural data fields of the one or more other data structures having a data type, each of the plural data fields of the one or more other data structures having a content type, the one or more other data structures being different in structure from the first data structure;
selecting desired source data for the plurality of patients from the plural data fields of the one or more other data structures;
assigning selected source data from the plural data fields of the one or more other data structures to the multiple data fields of the first data structure to populate the patient data of the first data structure,
wherein the assigning includes transforming source data of different content types for two or more fields of the one or more other data structures into a particular transformative data field of the first data structure,
wherein the multiple data fields of the first data structure include data fields for multiple treatment cycles at different times; and
processing the patient data of the first data structure for visualization at a graphical user interface such that representations of fields of the multiple data fields of the first data structure for the given patient are arranged for display via a single window of the graphical user interface, and such that certain fields of the multiple fields of the first data structure are structured so as to not visibly convey numerical or textual data via the graphical user interface, wherein at least some of the certain fields convey substantive information by virtue of not displaying visible numerical or textual data.

18. The non-transitory computer readable medium of claim 17, wherein assigning selected source data from the plural data fields comprises not assigning source data from some of the plural data fields of the one or more data structures.

19. The non-transitory computer readable medium of claim 17, wherein the multiple data fields of the first data structure for a given patient include a first set of data fields for storing screening information for the given patient, a second set of data fields for storing treatment information for the given patient, a third set of data fields for storing information regarding patient response to treatment for the given patient, a fourth set of data fields for storing laboratory results information for the given patient, a fifth set of data fields for storing medication information for the given patient, and a sixth set of data fields for storing adverse event information for the given patient; and
wherein the second, third, fourth, fifth, and sixth sets of data fields include data fields for multiple treatment cycles at different times; and

[symbol]

20. The non-transitory computer readable medium of claim 17, wherein after said assigning, some of the multiple fields of the first data structure are blank data fields containing no numerical or textual data, wherein at least some of the blank data fields convey substantive information such that patient data corresponding to such fields are within a normal range or unremarkable by virtue of being represented a blank data fields, and wherein some other fields of the first data structure are processed to be visualized at the graphical user interface with a non-numeric, non-textual character(s) to convey that source data are missing or absent for such fields.

21. The non-transitory computer readable medium of claim 17, wherein after said assigning, some of the multiple fields of the first data structure are blank data fields for which source data are absent or missing, and wherein at least some other fields of the first data structure are processed to be visualized at the graphical user interface with non-numeric, non-textual characters which convey substantive information such that patient data corresponding to such fields are within a normal range or unremarkable by virtue of being represented by non-numeric, non-textual characters.

22. The non-transitory computer readable medium of claim 17, the first data structure comprising the patient data for the plurality of patients is reduced in size by at least 50% compared to the one or more data structures comprising the source data for the plurality of patients, said reduction in size being achieved without additional computerized data compression algorithms.

23. The non-transitory computer readable medium of claim 17, wherein the one or more data structures comprises multiple data structures, and wherein said assigning comprises merging the source data of the multiple data structures into a second data structure configured as a two dimensional array.

24. The non-transitory computer readable medium of claim 17, wherein the computer instructions are configured to cause the processing system to not select for assignment source data from the one or more other data structures that comprises laboratory test data with a normal range.

25. A non-transitory computer memory storing a transformative computer data structure for facilitating efficient visualization of patient data, the data structure comprising:
multiple data fields in which to store patient data for a plurality of patients, the multiple data fields of the data structure including
a first set of data fields for storing screening information for a given patient,
a second set of data fields for storing treatment information for the given patient,
a third set of data fields for storing information regarding patient response to treatment,
a fourth set of data fields for storing laboratory results information for the given patient,
a fifth set of data fields for storing medication information for the given patient, and
a sixth set of data fields for storing adverse event information for the given patient,
wherein the second, third, fourth, fifth, and sixth sets of data fields include data fields for multiple treatment cycles,
wherein some data fields of the multiple data fields are configured to store patient data of two or more content types in a single data field,
wherein the second, third, fourth, fifth and sixth sets of data fields include data fields for multiple treatment cycles, and
wherein certain fields of the multiple fields of the first data structure are structured so as to not visibly convey numerical or textual data via the graphical user interface, wherein at least some of the certain fields convey substantive information by virtue of not displaying visible numerical or textual data.

26. The non-transitory computer memory of claim 25, wherein some of the multiple fields are blank data fields containing no numerical or textual data, wherein at least some of the blank data fields convey substantive information such that patient data corresponding to such fields are within a normal range or unremarkable by virtue of being represented a blank data fields, and wherein some other fields of the first data structure are processed to be visualized at the graphical user interface with a non-numeric, non-textual character(s) to convey that source data are missing or absent for such fields.

27. The non-transitory computer memory of claim 25, wherein after said assigning, some of the multiple fields of the first data structure are blank data fields for which source data are absent or missing, and wherein at least some other fields of the first data structure are processed to be visualized at the graphical user interface with non-numeric, non-textual characters which convey substantive information such that patient data corresponding to such fields are within a normal range or unremarkable by virtue of being represented by non-numeric, non-textual characters.
